(19) United States
(12) Patent Application Publication
(10) Pub. No.: US 2022/0389474 A1
(43) Pub. Date: Dec. 8, 2022

(54) MICROBIAL HOST CELLS FOR THE PRODUCTION OF HETEROLOGOUS CYANURIC ACID HYDROLASES AND BIURET HYDROLASES

(71) Applicant: DANISCO US INC., Palo Alto, CA (US)

(72) Inventors: Sharief Barends, Voorschoten (NL); Cristina Bongiorni, Fremont, CA (US); Feng Guo, Los Altos, CA (US); Lori Ann Maggio-Hall, Wilmington, DE (US); Lawrence Philip Wackett, St. Paul, MN (US); Gregory M. Whited, Palo Alto, CA (US)

(21) Appl. No.: 17/770,269

(22) PCT Filed: Oct. 14, 2020
(86) PCT No.: PCT/US20/55489

§ 371 Date: Apr. 19, 2022

Related U.S. Application Data
(60) Provisional application No. 62/926,665, filed on Oct. 28, 2019.

(51) Int. Cl. C12P 21/02; C12N 9/86; C12N 9/80; C12N 1/20

(52) U.S. Cl. CPC C12P 21/02; C12N 9/86; C12Y 305/01084; C12N 9/80; C12Y 305/02015; C12N 1/20

(57) ABSTRACT

The present disclosure is generally related to the fields of biology, molecular biology, genetics, microbial host cells, industrial enzyme production, and the like. More particularly, certain embodiments of the disclosure are related to microbial host cells for the production of heterologous proteins, which microbial host cells are well-suited for growth in submerged cultures for the large-scale production of heterologous cyanuric acid hydrolases and biuret hydrolases.

### CROSS REFERENCE TO RELATED APPLICATIONS

This application claims benefit to U.S. Provisional Patent Application No. 62/926,665, filed Oct. 28, 2019, which is incorporated herein by referenced in its entirety.

### TECHNICAL FIELD

The present disclosure is generally related to the fields of biology, molecular biology, genetics, microbial host cells, industrial enzyme production, and the like. More particularly, certain embodiments of the disclosure are related to microbial host cells for the production of heterologous proteins. More specifically, as presented and described herein, the microbial host cells of the disclosure are well-suited for growth in submerged cultures for the large-scale production of heterologous cyanuric acid hydrolases and biuret hydrolases.

### REFERENCE TO A SEQUENCE LISTING

The contents of the electronic submission of the text file Sequence Listing, named “NB41663-WO-PCT_Sequence_Listing.txt” was created on Oct. 6, 2020 and is 34 KB in size, which is hereby incorporated by reference in its entirety.

### BACKGROUND OF THE DISCLOSURE

The production of proteins (e.g., enzymes, antibodies, receptors, peptides, etc.) in microbial host cells are of particular interest in the biotechnological arts. For example, over the last thirty years the biopharmaceutical industry has introduced more than two hundred twenty (220) protein biologics (e.g., antibodies, protein/peptide therapeutics, etc.) to the market, of which about half of all approved recombinant protein therapeutics are produced using an Escherichia coli based expression system (Walsh, 2010). The implementation and use of E. coli based expression systems for recombinant protein production are often related to the genetic amenability of E. coli strains, which are generally known in the art to be easily modified, transformed, maintained, cultivated and the like.

However, as understood by one of skill in the art, E. coli expression systems have certain limitations, some of which are related to the lipopolysaccharide (LPS) present in the E. coli (Gram-negative) outer membrane. For example, in mammalian hosts (e.g., humans) these LPS are known endotoxins, which can induce a pyrogenic response and septic shock. Thus, any contaminating LPS must be removed from the recombinant protein product expressed in E. coli before it is considered safe for human use (Mamat et al., 2015). As described in Mamat et al., the removal of endotoxin from recombinant therapeutics and the testing to demonstrate endotoxin levels below a minimal threshold requires considerable effort, adding significant developmental and manufacturing costs. More particularly, the LPS removal costs are generally accepted in the protein biologics (therapeutics) arts, as the development and manufacturing costs of removing LPS from the recombinant protein is are typically offset or recaptured in the price charged for such protein biologic products. In this regard, the removal of LPS is such an issue, recent intellectual property has appeared (e.g., see U.S. Pat. No. 8,303,964) dealing with deletions in E. coli which lower the endotoxin expression in E. coli. Although the LPS response is lowered, it is not completely removed. In addition, the deletions that accomplish this lower response causes a significant repression to the growth and robustness of the cells when stressed with high protein expression or production of small molecules. This work further exemplifies the need in the field to find alternative host production systems which have no potential to produce LPS (e.g., non-Gram negative bacterial systems, or eukaryotic cells which intrinsically have no potential to produce LPS).

In contrast, industrial proteins (e.g., amylases, biuret hydrolases, catalases, cellulases, cyanuric acid hydrolases, glucoamylases, lipases, proteases and the like) recombinantly expressed in microbial cells are typically sold at much lower cost premiums relative to human biologic/therapeutic proteins, and as such, the development/manufacturing costs of removing LPS from recombinantly produced industrial proteins become burdensome and cost prohibitive in such industrial protein markets. For example, cyanuric acid hydrolases (EC 3.5.2.15) and biuret hydrolases (EC 3.5.1.84) are industrial enzymes used to remediate high cyanuric acid levels in aqueous liquids (e.g., such as swimming pool water, hot tub water, treated waste water and the like).

For example, to obtain sufficient quantities of isolated/purified CAH and BH enzymes for use in remediating CYA, the genes are typically cloned from the native (parental) source organism, and expressed recombinantly in E. coli cells, wherein the CAH or BH produced is isolated/purified therefrom (e.g., due to the general state of genetic proficiency and sophistication of E. coli expression systems vis-a-vis other microbial expression systems). As generally described in art, the genes encoding CAH family members are PCR amplified and cloned into a pET28b+ vector using NdeI and HindIII restriction sites, wherein the resulting vectors are transformed into either E. coli BL21(DE3) or E. coli BL21(DE3) pLys, and the His⁺ tagged CAH enzymes are purified therefrom (e.g., using a 5 ml HiTrap chelating HP column). As described in the art (e.g., see PCT Publication No. WO2007/107981), the recombinant His⁺ tagged CAH enzyme was characterized by a Vmax of 0.3 mmoles/min/mg, suggesting that for commercial development, a source of CAH enzyme with a higher Vmax would be an advantage.

Thus, certain unmet needs in the art are related to isolated cyanuric acid hydrolases and isolated biuret hydrolases for use in remediating CYA in swimming pool water, hot tub water and the like. Certain other unmet needs in the art are related to host cell expression systems for the heterologous production of CAH enzymes and BH enzymes, and more particularly, the cost effective production of such CAH enzymes and BH enzymes at industrial scales for use in CYA remediation processes. Certain other unmet needs in the art are therefore related to the heterologous expression/production of CAH and BH enzymes in microbial host systems which do not produce LPS endotoxins, particularly excluding Gram-negative host expressions systems (e.g., E. coli) comprising LPS in their outer membrane. Other unmet needs in the art are related to the isolation, recovery, purification and the like of such heterologously produced CAH and BH enzymes.

As set forth and described hereinafter, the instant disclosure addresses various needs in the art related to cyanuric acid hydrolases and biuret hydrolases for use in remediating CYA in swimming pool water, hot tub water and the like. As described hereinafter, the microbial (host) cells of the instant disclosure are particularly well-suited for growth in submerged cultures for the large-scale production of heterologous CAH and BH proteins.

### SUMMARY OF THE DISCLOSURE

Certain embodiments of the instant disclosure are related to the microbial (host) cells for the large-scale production of heterologous cyanuric acid hydrolases and biuret hydrolases. Thus, certain embodiments are related to microbial host cells comprising a heterologous polynucleotide encoding a cyanuric acid hydrolase (CAH) and/or comprising a heterologous polynucleotide encoding a heterologous biuret hydrolase (BH). In certain embodiments, a heterologous polynucleotide encoding the CAH is derived from a parental cell selected from the group consisting of M. thermoacetica, B. diazoefficiens, Bradyrhizobium sp. (WSM1253), Pseudolabrys sp. (Root1462) and A. citrulli (122227) and/or wherein the heterologous polynucleotide encoding the BH is derived from a Herbaspirillum sp. (BH-1) parental cell. In other embodiments, a CAH encoded by a heterologous polynucleotide comprises about 50% or greater amino acid sequence identity to any one of SEQ ID NO: 2, SEQ ID NO: 6, SEQ ID NO: 8, SEQ ID NO: 10 and SEQ ID NO:12. In other embodiments, a CAH encoded by a heterologous polynucleotide is an EC 3.5.2.15 enzyme. In another embodiment, a BH encoded by a heterologous polynucleotide is an EC 3.5.1.84 enzyme. In other embodiments, a CAH encoded by a heterologous polynucleotide comprises SEQ ID NO: 23.

In certain other embodiments, a host cell of the disclosure is a prokaryotic host cell or a eukaryotic microbial host cell. In other embodiments, a prokaryotic host cell of the disclosure excludes Gram-negative bacterial cells. In other embodiments, a host cell of the disclosure is selected from the group consisting of a Gram-positive bacterial cell, a filamentous fungal cell and a yeast cell.

In certain other embodiments, a heterologous polynucleotide encoding the CAH is a polynucleotide expression cassette and/or a heterologous polynucleotide encoding the BH is a polynucleotide expression cassette.

In other embodiments, a host cell of the disclosure is fermented under suitable conditions to express and produce the heterologous CAH and/or to express and produce the heterologous BH. In other embodiments, the heterologous CAH expressed and produced is isolated from the host cell, or the host cell fermentation broth. In other embodiments, the heterologous BH expressed and produced is isolated from the host cell, or the host cell fermentation broth.

Thus, certain other embodiments are directed to isolated CAH proteins and/or an isolated BH proteins produced by host cells of the disclosure. In other embodiments, an isolated CAH and/or isolated BH produced by a host cell of the disclosure comprises no detectable lipopolysaccharides (LPS).

In other embodiments, an isolated CAH produced by a host cell of the disclosure comprises equivalent, or increased activity, relative to the activity of same CAH endogenously produced and isolated from a parental cell from which the CAH gene was derived. In other embodiments, an isolated CAH produced by a host cell of the disclosure comprises equivalent, or increased activity, relative to the activity of same CAH heterologously expressed and isolated from an E. coli host cell. In other embodiments, an isolated CAH produced by a host cell of the disclosure does not comprise a poly-histidine tag.

In certain other embodiments, an isolated BH produced by a host cell of the disclosure comprises equivalent, or increased activity, relative to the activity of same BH endogenously produced and isolated from a parental cell from which the BH gene was derived. In other embodiments, an isolated BH produced by a host cell of the disclosure comprises equivalent, or increased activity, relative to the activity of same BH heterologously expressed and isolated from an E. coli host cell.

In other embodiments, the disclosure is related to methods for expressing and producing a heterologous CAH and/or a heterologous (BH) in a host cell, comprising (a) obtaining a suitable host cell and introducing into the host cell a polynucleotide expression cassette encoding a heterologous CAH and/or introducing into the host cell a polynucleotide expression cassette encoding a heterologous BH, (b) fermenting the host cell under suitable conditions for the expression and production of the heterologous CAH and/or the heterologous BH, and (c) isolating the heterologous CAH and/or heterologous BH from the host cell or the host cell fermentation broth. In certain embodiments, the gene encoding the CAH is derived from a parental cell selected from the group consisting of M. thermoacetica, B. diazoefficiens, Bradyrhizobium sp. (WSM1253), Pseudolabrys sp. (Root1462) and A. citrulli (122227) and/or wherein the heterologous polynucleotide encoding the BH is derived from a Herbaspirillum sp. (BH-1) parental cell. In other embodiments, a gene (or ORF) encoding a CAH comprises about 50% or greater amino acid sequence identity to any one of SEQ ID NO: 2, SEQ ID NO: 6, SEQ ID NO: 8, SEQ ID NO: 10 and SEQ ID NO:12.

In other embodiments of the methods, a heterologous CAH expressed and produced is isolated from the host cell, or the host cell fermentation broth. In other embodiments, a heterologous BH expressed and produced is isolated from the host cell or the host cell fermentation broth. In certain embodiments, a host cell of the disclosure is a GRAS microbial host cell. In other embodiments, a GRAS microbial host cell does not include (does not comprise) an antibiotic resistance marker (marker-free).

Certain embodiments are therefore related an isolated CAH and/or an isolated BH produced by the methods of the disclosure. In other embodiments of the methods, an isolated CAH and/or an isolated BH comprises no detectable lipopolysaccharides (LPS).

In certain other embodiments of the methods, an isolated CAH produced thereby comprises equivalent, or increased activity relative to the activity of same CAH endogenously produced and isolated from a parental cell from which the CAH gene was derived. In other embodiments, an isolated CAH produced thereby comprises equivalent, or increased activity, relative to the activity of same CAH heterologously expressed and isolated from an E. coli host cell.

In certain other embodiments of the methods, an isolated BH produced thereby comprises equivalent, or increased activity relative to the activity of same BH endogenously produced and isolated from a parental cell from which the BH gene was derived. In certain other embodiments of the methods, an isolated BH produced thereby comprises equivalent, or increased activity, relative to the activity of same BH heterologously expressed and isolated from an E. coli host cell.

### BRIEF DESCRIPTION OF THE BIOLOGICAL SEQUENCES

SEQ ID NO: 1 is a nucleic acid sequence encoding a M. thermoacetica (ATCC 39703) cyanuric acid hydrolase (CAH) of SEQ ID NO: 2.

SEQ ID NO: 2 is the amino acid sequence of the M. thermoacetica (ATCC 39703) cyanuric acid hydrolase (CAH).

SEQ ID NO: 3 is a nucleic acid sequence encoding a M. thermoacetica (ATCC 39703) C46A variant cyanuric acid hydrolase (CAH) of SEQ ID NO: 4.

SEQ ID NO: 4 is the amino acid sequence of the variant (C46A) M. thermoacetica CAH.

SEQ ID NO: 5 a nucleic acid sequence encoding a Bradyrhizobium diazoefficiens (USDA 110) cyanuric acid hydrolase (CAH) of SEQ ID NO: 6, which nucleic acid sequence has been codon-optimized for expression in a Bacillus sp. host cell.

SEQ ID NO: 6 is the amino acid sequence of the B. diazoefficiens (USDA 110) CAH.

SEQ ID NO: 7 is a nucleic acid sequence encoding a Bradyrhizobium sp. (WSM1253) cyanuric acid hydrolase (CAH) of SEQ ID NO: 8, which nucleic acid sequence has been codon-optimized for expression in a Bacillus sp. host cell.

SEQ ID NO: 8 is the amino acid sequence of the Bradyrhizobium sp. (WSM1253) CAH.

SEQ ID NO: 9 is a nucleic acid sequence encoding a Pseudolabrys sp. (Root1462) cyanuric acid hydrolase (CAH) of SEQ ID NO: 10, which nucleic acid sequence has been codon-optimized for expression in a Bacillus sp. host cell.

SEQ ID NO: 10 is the amino acid sequence of the Pseudolabrys sp. (Root1462) CAH.

SEQ ID NO: 11 is a nucleic acid sequence encoding a Acidovorax citrulli (122227) cyanuric acid hydrolase (CAH) of SEQ ID NO: 12, which nucleic acid sequence has been codon-optimized for expression in a Bacillus sp. host cell.

SEQ ID NO: 12 is the amino acid sequence of the A. citrulli (122227) CAH.

SEQ ID NO: 13 is a nucleic acid sequence encoding a Herbaspirillum sp. (BH-1) biuret hydrolase (BH) of SEQ ID NO: 14, which nucleic acid sequence has been codon-optimized for expression in a Herbaspirillum rubris cell.

SEQ ID NO: 14 is the amino acid sequence of the Herbaspirillum sp. BH.

SEQ ID NO: 15 is a synthetic primer sequence CAH001.

SEQ ID NO: 16 is a synthetic primer sequence CAH002.

SEQ ID NO: 17 is a synthetic primer sequence CAH003.

SEQ ID NO: 18 is a synthetic primer sequence CAH004.

SEQ ID NO: 19 is a synthetic primer sequence CAH005.

SEQ ID NO: 20 is a synthetic primer sequence CAH006.

SEQ ID NO: 21 is a synthetic primer sequence CAH007.

SEQ ID NO: 22 is a synthetic primer sequence CAH008.

SEQ ID NO: 23 is an artificial amino acid (consensus) sequence.

### BRIEF DESCRIPTION OF THE DRAWINGS

FIG. 1 presents the chemical structures of cyanuric acid (triol/trione) tautomers (FIG. 1A), 1,3,5-Trichloro-1,3,5-triazinane-2,4,6-trione or “Trichlor” (FIG. 1B), 1,3-dichloro-1,3,5-triazinane-2,4,6-trione or “Dichlor” (FIG. 1C) and a schematic diagram of the enzymatic hydrolysis (e.g., AtzD) of cyanuric acid to biuret (FIG. 1D). As presented in FIG. 1A, the cyanuric (triol) is labeled 1 and the isocyanuric (trione) is labeled 2. As presented in FIG. 1D, cyanuric acid is shown on the left of the arrow, the cyanuric acid hydrolase (CAH; above the arrow) and the biuret product is shown on the right of the arrow. As presented in FIG. 1E, biuret is shown on the left of the arrow, the biuret hydrolase (BH; above the arrow) and the allophonate product is shown on the right of the arrow.

FIG. 2 shows the activities of CAH homologues heterologously expressed and purified from Bacillus subtilis host cells. For example, as shown in FIG. 2, the six (6) heterologously expressed and purified CAH enzymes described were incubated in simulated swimming pool water containing 200 ppm CYA and incubated at room temperature overnight. The amount of CAH used in the experiment was empirically determined to show that the CAH proteins are active, but do not reflect the specific activity of each protein. The amount of CYA consumed was calculated by subtracting the remaining CYA from the starting 200 ppm.

FIG. 3 presents a conserved cyanuric acid hydrolase primary (1°) amino acid sequence (SEQ ID NO: 23) set forth and described below in Example 5. As shown in FIG. 3 (SEQ ID NO: 23), the amino acid “Xaa” in the first (1) position is a “Tyr” or “Phe”, the amino acid “Xaa” in the second (2) position may be any amino acid, the amino acid “Xaa” in the sixth (6) position may be any amino acid, the amino acid “Xaa” in the eight (8) position is a “His” or “Asn”, the amino acid “Xaa” in the twelfth (12) position may be any amino acid, and the amino acid “Xaa” in the six-tenth (16) position is a “Pro” or “Ser”.

FIG. 4 shows the primary amino acid sequences of proteins empirically confirmed to comprise CAH activity and having an exact match to the full length consensus amino acid consensus sequence of SEQ ID NO: 23. For example, the C-terminal amino acid residues of these proteins comprising CAH activity are presented as bold underlined residues in FIG. 4, wherein these emphasized residues are an exact match to the full length consensus amino acid consensus sequence of SEQ ID NO: 23.

FIG. 5 shows the primary amino acid sequences of proteins which do not comprise CAH activity (e.g., see Example 5). As indicated in FIG. 5, the C-terminal residues of these proteins lacking CAH activity do not have an exact match to the full length consensus amino acid consensus sequence of SEQ ID NO: 23.

FIG. 6 presents the activity of CAH proteins (SEQ ID NO: 6 and SEQ ID NO: 10) expressed/produced by Saccharomyces cerevisiae host cells (i.e., exemplary yeast cells) and Trichoderma reesei host cells (i.e., exemplary filamentous fungus cells).

### DETAILED DESCRIPTION

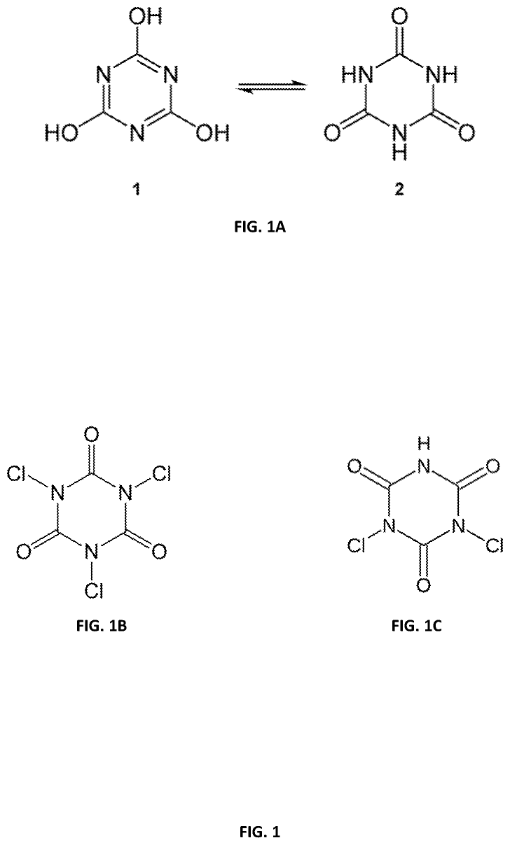

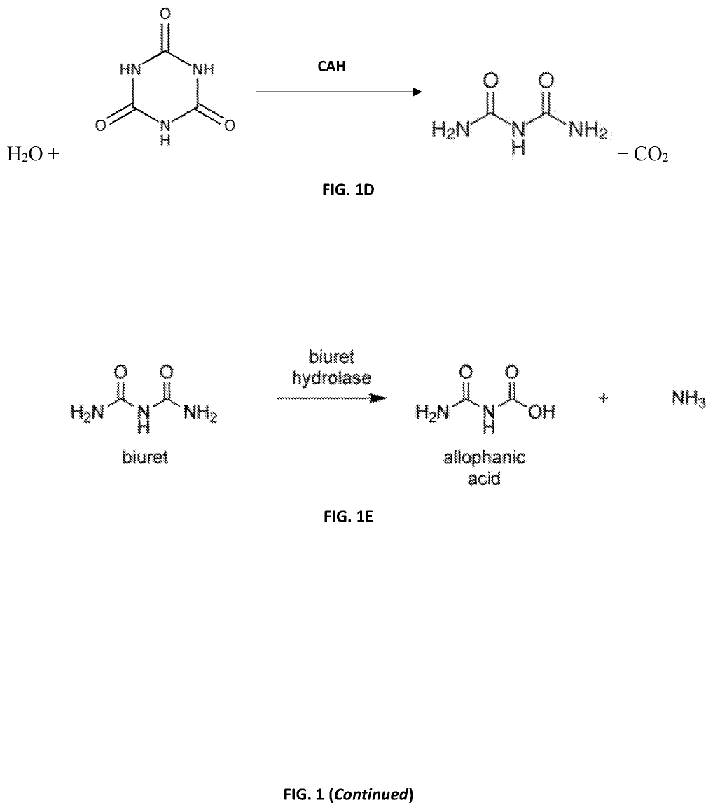

As generally described in the art (see, PCT Publication No. WO2007/107981, PCT Publication No. WO2016/141026 and Seffernik et al., 2012), cyanuric acid hydrolase (CAH) and biuret hydrolase (BH) enzymes are used to remediate cyanuric acid in aqueous liquids via catalytic breakdown of cyanuric acid to biuret and catalytic breakdown of biuret to allophonate, respectively (e.g., FIG. 1D and FIG. 1E, respectively). As presented and exemplified herein, the instant disclosure addresses ongoing and unmet needs in the art related CYA remediation in aqueous liquids. Certain embodiments of the disclosure are therefore related to isolated (purified) cyanuric acid hydrolases and isolated (purified) biuret hydrolases for use in remediating CYA in swimming pool water, hot tub water and the like. For example, as briefly set forth in the Background section, all currently known CAH and BH enzymes recombinantly or heterologously produced for use in academic and/or industrial research for the intended use in remediating CYA have been expressed in E. coli (Gram-negative) host cell systems, including use of these whole cells or the enzymes isolated/purified therefrom.

As set forth and described herein, certain embodiments of the disclosure are therefore related to the construction and optimization of microbial host expression systems for the production of (heterologous) CAH and/or (heterologous) BH enzymes. Certain embodiments are related to microbial host cells generally recognized as safe (GRAS) and more particularly, the genetic modification of such GRAS host cells for the enhanced production of (heterologous) CAH and BH enzymes therefrom. Thus, the microbial (host) cells described and exemplified herein are particularly well-suited for growth in submerged cultures for the large-scale production of heterologous CAH and BH enzymes.

Certain other embodiments are related to the isolated CAH and/or BH enzymes expressed/produced by a microbial host cell of the disclosure. Certain embodiments are therefore related to the isolation, recovery, purification and the like of such CAH and/or BH enzymes expressed/produced by a microbial host cell of the disclosure.

In certain embodiments, a CAH enzyme expressed/produced by a microbial host cell of the disclosure does not include or comprise a poly-histidine (His⁺) tag.

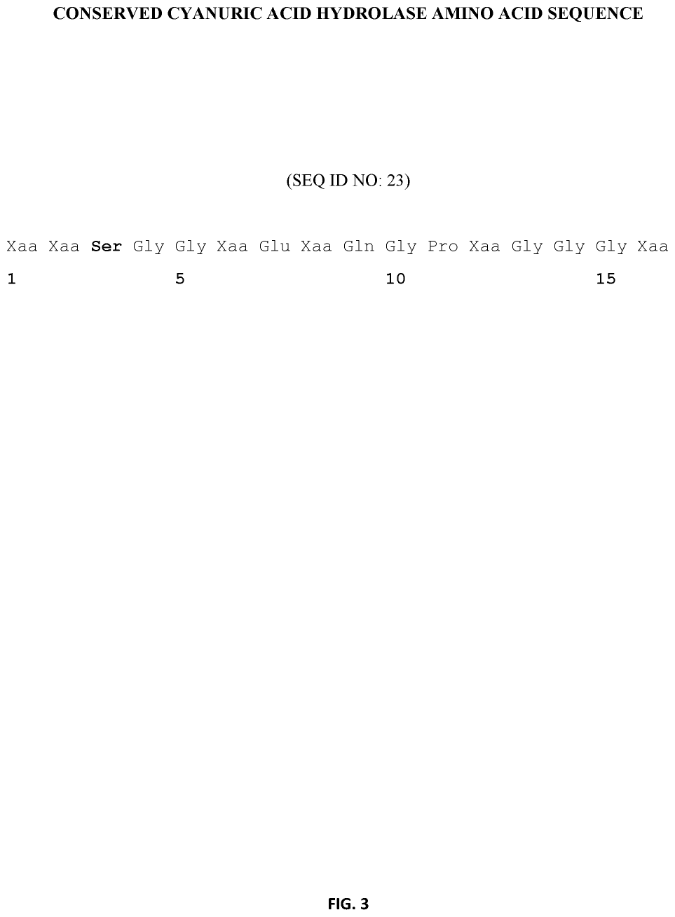

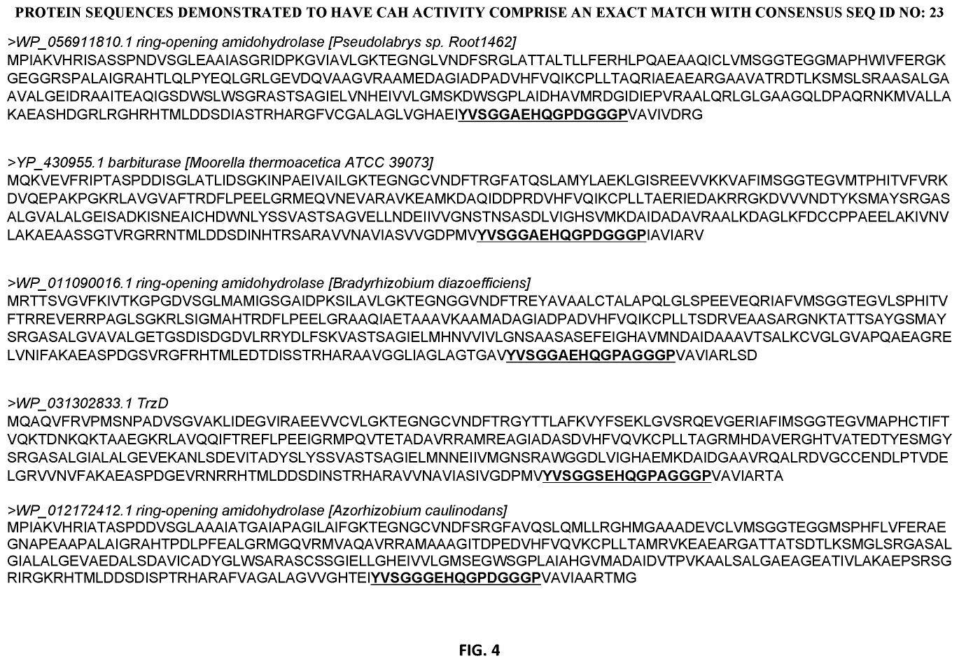

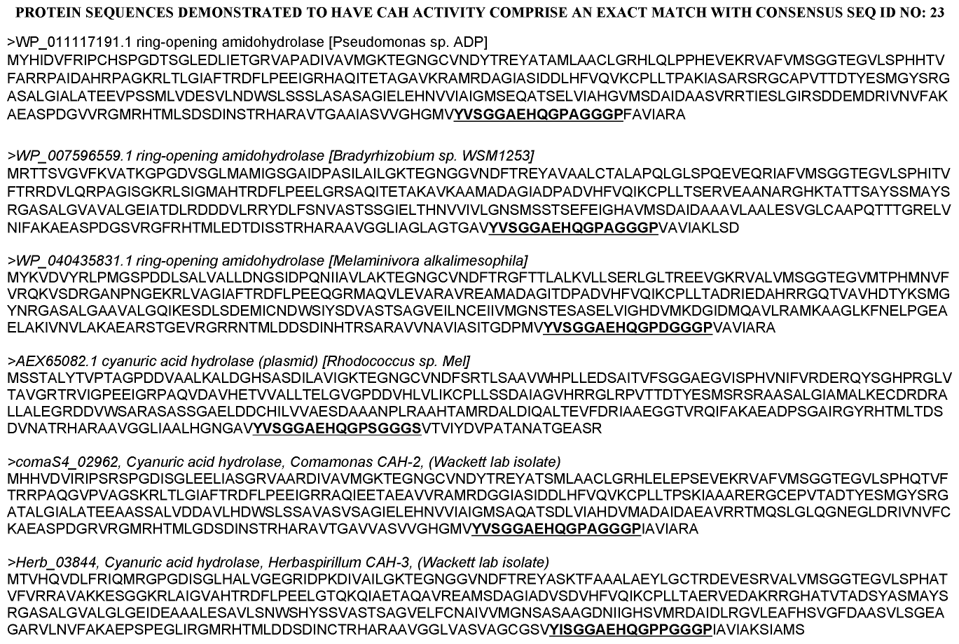

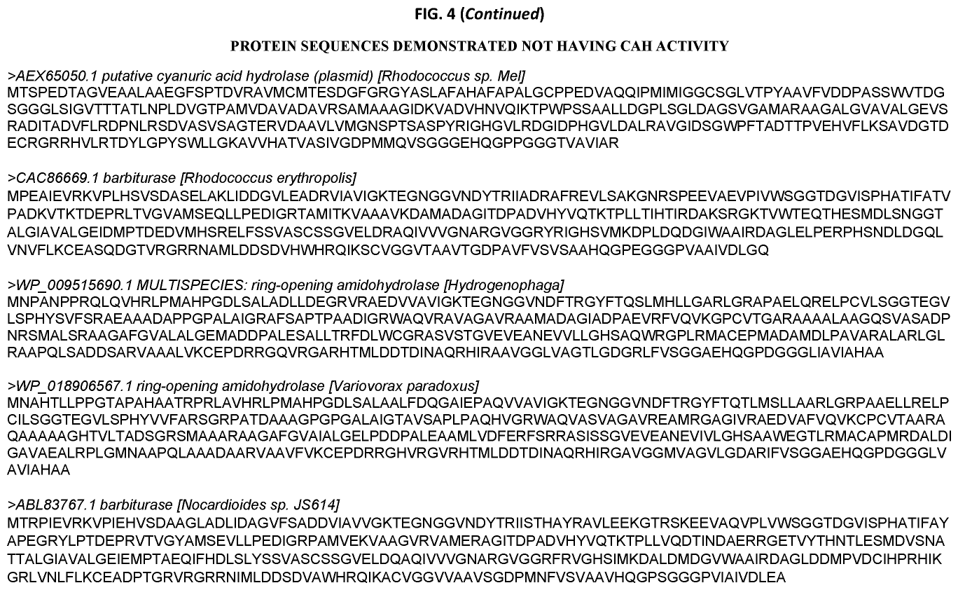

In certain embodiments, a novel sixteen (16) amino acid consensus sequence of SEQ ID NO: 23 is used to identify, screen, and/or verify protein (amino acid) sequences comprising CAH activity. More particularly, as described below in Example 5, Applicant identified a novel sixteen (16) amino acid consensus sequence set forth in SEQ ID NO: 23 (FIG. 3), which is near the C-terminus of the cyanuric acid hydrolase protein (e.g., see, FIG. 4, bold underlined residues).

The CAH consensus sequence (SEQ ID NO: 23) includes one of the conserved serine (Ser) residues in the CAH active site, and further includes amino acid (residues) in the region of the bound metal residue that has been identified in the X-ray structures of all cyanuric acid hydrolase to date (Bera et al., 2017; Shi et al., 2019).

Thus, in certain embodiments, a CAH enzyme expressed/produced by a microbial host cell of the disclosure does not include or comprise a C-terminal poly-histidine (His⁺) tag. For example, poly-histidine (His⁺) tags are often used for affinity purification of His⁺-tagged recombinant proteins expressed in E. coli and other prokaryotic expression systems. Without wishing to be bound to any particular theory, Applicant contemplates herein that poly-histidine tagged CAH enzymes (i.e., proteins comprising CAH activity) and uses thereof are generally subjected to losses in enzymatic activity and/or stability, relative to CAH enzymes (i.e., proteins comprising CAH activity) which do not include or comprise a poly-histidine (His⁺) tag.

Certain other embodiments are related to microbial host cells of the disclosure comprising an introduced polynucleotide (e.g., an expression cassette) encoding a heterologous CAH and/or an introduced polynucleotide encoding a heterologous BH. In certain embodiments, a microbial host cell ofthe disclosure (e.g., comprising an introduced polynucleotide (expression cassette) encoding a heterologous CAH and/or BH) does not comprise an antibiotic resistance marker.

In certain embodiments, a microbial host cell of the disclosure produces an increased amount of a (heterologous) CAH relative to a parental microbial cell (source organism) from which the wild-type (endogenous) CAH gene was derived. In other embodiments, a microbial host cell produces an increased amount of a (heterologous) BH relative to a parental microbial cell (source organism) from which the wild-type (endogenous) BH gene was derived. In other embodiments, a microbial host cell of the disclosure produces an increased amount of a (heterologous) CAH relative to an E. coli host cell comprising and expressing the same (heterologous) CAH. Thus, in certain other embodiments, a microbial host cell of the disclosure produces an increased amount of a (heterologous) BH relative to an E. coli host cell comprising and expressing the same (heterologous) BH.

In certain other embodiments, an isolated (heterologous) CAH produced by a microbial host cell of the disclosure comprises equivalent or enhanced activity relative to the same CAH enzyme isolated from its parental source. In other embodiments, an isolated (heterologous) BH produced by a microbial host cell of the disclosure comprises equivalent or enhanced activity relative to the same BH enzyme isolated from its parental source. In certain other embodiments, an isolated (heterologous) CAH produced by a microbial host cell of the disclosure comprises equivalent or enhanced activity relative to the same CAH heterologously expressed and isolated from an E. coli cell. In other embodiments, an isolated (heterologous) BH produced by a microbial host cell of the disclosure comprises equivalent or enhanced activity relative to the same BH heterologously expressed and isolated from an E. coli cell. In certain other embodiments, CAH or BH is advantaged for purification with respect to Gram-negative cell production, purification, and recovery. In certain embodiments, an isolated (heterologous) CAH or an isolated (heterologous) BH expressed/produced by a microbial host cells of the disclosure comprises no detectable LPS endotoxin.

Thus, as described hereinafter, the microbial cells of the instant disclosure are particularly well-suited for growth in submerged cultures for the large-scale production of heterologous cyanuric acid hydrolases and biuret hydrolases.

### I. Definitions

Prior to describing the present compositions and methods in detail, the following terms are defined for clarity. Terms not defined should be accorded their ordinary meanings as used in the relevant art. Unless defined otherwise, all technical and scientific terms used herein have the same meaning as commonly understood by one of ordinary skill in the art to which the present compositions and methods apply.

Unless defined otherwise, all technical and scientific terms used herein have the same meaning as commonly understood by one of ordinary skill in the art to which the present compositions and methods apply. Although any methods and materials similar or equivalent to those described herein can also be used in the practice or testing of the present compositions and methods, representative illustrative methods and materials are now described. All publications and patents cited herein are incorporated by reference in their entirety.

It is further noted that the claims may be drafted to exclude any optional element. As such, this statement is intended to serve as antecedent basis for use of such exclusive terminology as “solely,” “only”, “excluding”, “not including” and the like, in connection with the recitation of claim elements, or use of a “negative” limitation or proviso thereof.

As will be apparent to those of skill in the art upon reading this disclosure, each of the individual embodiments described and illustrated herein has discrete components and features which may be readily separated from or combined with the features of any of the other several embodiments without departing from the scope or spirit of the present compositions and methods described herein. Any recited method can be carried out in the order of events recited or in any other order which is logically possible.

As used herein, the “genus Bacillus” or “Bacillus sp.” cells for manipulation, construction and use as described herein include all species within the genus “Bacillus”’ as known to those of skill in the art, including but not limited to B. subtilis, B. licheniformis, B. lentus, B. brevis, B. stearothermophilus, B. alkalophilus, B. amyloliquefaciens, B. clausii, B. halodurans, B. megaterium, B. coagulans, B. circulars, B. lautus, and B. thuringiensis. It is recognized that the genus Bacillus continues to undergo taxonomical reorganization. Thus, it is intended that the genus include species that have been reclassified, including but not limited to such organisms as B. stearothermophilus, which is now named “Geobacillus stearothermophilus”.

As used herein, “filamentous fungal” cells for manipulation, construction and use as described herein are generally from the phylum Ascomycota, subphylum Pezizomycotina, particularly fungi that have a vegetative hyphae state. Such organisms include filamentous fungus cells used for the production of commercially important industrial and pharmaceutical proteins, including, but not limited to Trichoderma sp., Aspergillus sp Fusarium sp., Penicillium sp., Chrysosporium sp., Cephalosporium sp., Talaromyces sp., Geosmithia sp., Neurospora sp., Myceliophthora sp. and the like. For example, in certain embodiments, filamentous fungus cells and strains thereof include, but are not limited to Trichoderma reesei (previously classified as Trichoderma longibrachiatum and Hypocrea jecorina), Aspergillus niger, Aspergillus fumigatus, Aspergillus itaconicus, Aspergillus oryzae, Aspergillus nidulans, Aspergillus terreus, Aspergillus sojae, Aspergillus japonicus, Neurospora crassa, Penicillium funiculosum, Penicillium chrysogenum, Talaromyces (Geosmithia) emersonii, Fusarium venenatum, Myceliophthora thermophila, Chrysosporium lucknowense (C1) and the like.

As used herein, “Aspergillus” or “Aspergillus sp,” includes all species within the genus Aspergillus as known to those of skill in the art, including but not limited to A. oryzae, A. niger, A. awamori, A. nidulans, A. sojae. A. japonicus, A. kawachi and A. aculeatus.

As used herein, the terms “yeast cells” or simply “yeast” refer to organisms from the phyla Ascomycota and Basidiomycota. Exemplary yeast is budding yeast from the order Saccharomycetales. Particular examples of yeast are Saccharomyces sp., including but not limited to S. cerevisiae. Yeast are therefore unicellular eukaryotic microorganisms including S. cerevisiae, Kluyveromyces sp., Lachancea sp. and Schizosaccharomyces sp.

As used herein, the phrase “microbial cell(s)” includes prokaryotic cells (i.e., Bacteria and Archaea) and eukaryotic (microbial) cells such as protists (e.g., algae and slime molds), green algae, fungi (e.g., yeast and filamentous fungi) and the like, as generally understood by one skilled in the art.

In certain embodiments, microbial cells of the disclosure exclude all Gram-negative bacteria. In other embodiments, microbial cells of the disclosure exclude the Gram-negative bacterium Escherichia coli. In certain other embodiments, microbial cells of the disclosure do not produce detectable lipopolysaccharides (LPS) endotoxin.

As used herein, “lipopolysaccharides”, abbreviated “LPS”, are glycolipid endotoxins found in the outer membrane of Gram-negative bacteria. The LPS endotoxin is known to bind the CD14/TLR4/MD2 receptor complex in many cell types, particularly monocytes, dendritic cells, macrophages and B cells, which promote the secretion of pro-inflammatory cytokines, nitric oxide, and eicosanoids. For example, it is desirable that such isolated CAH and/or BH enzymes for use in CYA remediation (e.g., swimming pool water, hot tub water) have no detectable LPS (endotoxins), and as such, are considered safe for human contact/exposure (e.g., as used or applied by residential pool owner and/or commercial pool technicians).

As used herein, an “endogenous gene” refers to a gene in its natural location in the genome of an organism.

As used herein, an “endogenous gene” encodes an “endogenous protein”.

As used herein, the phrase “native cell” and “parental cell” may be used interchangeably, and refer to a naturally occurring microbial cell of the disclosure comprising an endogenous gene encoding an endogenous cyanuric acid hydrolase and/or comprising an endogenous gene encoding an endogenous biuret hydrolase.

As used herein, a parental Moorella thermoacetica cell named “M. thermoacetica” comprises an endogenous gene (SEQ ID NO: 1) encoding a native cyanuric acid hydrolase (SEQ ID NO: 2), a parental Bradyrhizobium diazoefficiens cell named “B. diazoefficiens” comprises an endogenous gene (SEQ ID NO: 5) encoding a native cyanuric acid hydrolase (SEQ ID NO: 6), a parental Bradyrhizobium sp. cell named “Bradyrhizobium sp. (WSM1253)” comprises an endogenous gene (SEQ ID NO: 7) encoding a native cyanuric acid hydrolase (SEQ ID NO: 8), a parental Pseudolabrys sp. cell named “Pseudolabrys sp. (Root1462)” comprises an endogenous gene (SEQ ID NO: 9) encoding a native cyanuric acid hydrolase (SEQ ID NO: 10) and a parental Acidovorax citrulli cell named “A. citrulli (122227)” comprises an endogenous gene (SEQ ID NO: 11) encoding a native cyanuric acid hydrolase (SEQ ID NO: 12).

As noted herein, the parental cell “B. diazoefficiens” described above was originally named “Bradyrhizobium japonicum (USDA 110)”, Sugawara et al. (2017).

As used herein, a mutant of a parental M. thermoacetica cell named “M. thermoacetica (C46A)” comprises a mutated gene (SEQ ID NO: 3) encoding a variant cyanuric acid hydrolase (SEQ ID NO: 4) comprising a cysteine (C) to alanine (A) substitution at amino acid position 46 of SEQ ID NO: 4.

As used herein, a “heterologous gene” refers to a gene (or ORF) which is not normally found or naturally occurring in the host cell.

As used herein, a “heterologous gene” encodes a “heterologous protein”.

As used herein, a “heterologous protein” refers to a protein that is not normally produced by the host organism.

As used herein, the phrase “host cell” will be used when referring to a microbial cell of the disclosure comprising a heterologous gene (or ORF) encoding a cyanuric acid hydrolase and/or comprising a heterologous gene (or ORF) encoding a biuret hydrolase. Thus, a “host cell” has the capacity to act as a host or expression vehicle for a newly introduced gene or (ORF) sequence.

As used herein, “heterologous gene expression” or a “heterologous gene expression system” refer to the expression a heterologous gene (or part of a gene) in a host cell, which host cell does not naturally comprise the gene (or gene fragment). For example, insertion of the heterologous CAH or BH gene into the host cell may be performed by recombinant DNA technology.

As used herein, a “heterologous nucleic acid construct” or a “heterologous nucleic acid sequence” has a portion of the sequence which is not native to the host cell in which it is expressed.

As used herein, the term “ComK polypeptide” is defined as the product of a comK gene; a transcription factor that acts as the final auto-regulatory control switch prior to competence development; involved with activation of the expression of late competence genes involved in DNA-binding and uptake and in recombination (Liu and Zuber, 1998).

As used herein, the combined term “expresses/produces”, as used in phrases such as a host cell “expresses/produces” an increased amount of a cyanuric acid hydrolase and/or a host cell “expresses/produces” an increased amount of a biuret hydrolase”, the combined term “expresses/produces” is meant to include any steps involved in the expression and production of a cyanuric acid hydrolase and/or biuret hydrolase of the disclosure.

As used herein, the phrase a host cell expresses/produces an “increased amount of a (heterologous) CAH relative to the parental source”, the ‘increased amount’ of the heterologous CAH is relative to the expression/production of the same CAH (endogenously) expressed/produced in the parental cell from which the CAH gene was derived, wherein the host cell and the parental cell are cultivated under similar or identical conditions.

As used herein, the phrase a host cell expresses/produces an “increased amount of a (heterologous) BH relative to the parental source”, the ‘increased amount’ of the heterologous BH is relative to the expression/production of the same BH (endogenously) expressed/produced in the parental cell from which the BH gene was derived, wherein the host cell and the parental cell are cultivated under similar or identical conditions.

As used herein, the phrase a host cell expresses/produces an “increased amount of a (heterologous) CAH relative to an E. coli cell”, the ‘increased amount’ of the heterologous CAH is relative to the expression/production of the same (heterologous) CAH expressed/produced in an E. coli cell, wherein the host cell and the E. coli cell are cultivated under similar or identical conditions.

As used herein, the phrase a host cell expresses/produces an “increased amount of a (heterologous) BH relative to an E. coli cell”, the ‘increased amount’ of the heterologous BH is relative to the expression/production of the same (heterologous) BH expressed/produced in an E. coli cell, wherein the host cell and the E. coli cell are cultivated under similar or identical conditions.

As used herein, the phrase “cultivated under similar or identical conditions” indicates that the cells are grown/cultured/fermented under the same conditions (e.g., the same conditions such as media, temperature, pH and the like). In certain preferred embodiments, the cells are cultivated under aerobic conditions in submerged culture.

As used herein, the term “cyanuric acid” (abbreviated hereinafter, “CYA”) collectively refers to either of the CYA tautomers presented in FIG. 1A. For example, as shown in FIG. 1A, the cyanuric (triol) is labeled 1 and the isocyanuric (trione) is labeled 2. Thus, as used herein, the CYA tautomers shown in FIG. 1A may collectively be abbreviated as “C3N303” or simply “CYA”.

As used herein, the term “cyanuric acid hydrolase” may be abbreviated as “CAH”, and includes, but is not limited to, any enzyme comprising cyanuric acid hydrolase activity (e.g., see, FIG. 1D). In certain embodiments, a CAH comprises Enzyme Commission Number EC 3.5.2.15.

In certain other embodiments, an enzyme comprising CAH activity comprises about 35% to about 50% amino acid sequence identity to a CAH enzyme selected from the group consisting of SEQ ID NO: 2, SEQ ID NO: 4, SEQ ID NO: 6, SEQ ID NO: 8, SEQ ID NO: 10 and SEQ ID NO: 12.

In other embodiments, an enzyme comprising CAH activity is derived or obtained from a parental M. thermoacetica cell, a parental B. diazoefficiens cell, a parental Bradyrhizobium sp. (WSM1253) cell, a parental Pseudolabrys sp. (Root1462) cell or a parental A. citrulli (122227) cell.

In other embodiments, a protein (enzyme) comprising CAH activity comprises a consensus sequence set forth in SEQ ID NO: 23. In certain embodiments, a protein comprising CAH activity comprises a primary (1°) amino acid comprising SEQ ID NO: 23 near the C-terminus of the CAH protein, e.g., as shown in FIG. 4.

As used herein, the term “biuret hydrolase” may be abbreviated as “BH”, and includes, but is not limited to, any enzyme comprising biuret hydrolase activity (e.g., see, FIG. 1E). In certain embodiments, a BH comprises Enzyme Commission Number EC 3.5.1.84.

As used herein, “nucleic acid” refers to a nucleotide or polynucleotide sequence, and fragments or portions thereof, as well as to DNA, cDNA, and RNA of genomic or synthetic origin, which may be double-stranded or single-stranded, whether representing the sense or antisense strand. It will be understood that as a result of the degeneracy of the genetic code, a multitude of nucleotide sequences may encode a given protein. It is understood that the polynucleotides (or nucleic acid molecules) described herein include “genes”, “vectors” and “plasmids”.

Accordingly, the term “gene”, refers to a polynucleotide that codes for a particular sequence of amino acids, which comprise all, or part of a protein coding sequence, and may include regulatory (non-transcribed) DNA sequences, such as promoter sequences, which determine for example the conditions under which the gene is expressed. The transcribed region of the gene may include untranslated regions (UTRs), including introns, 5′-untranslated regions (UTRs), and 3′-UTRs, as well as the coding sequence.

As used herein, the term “coding sequence” refers to a nucleotide sequence, which directly specifies the amino acid sequence of its (encoded) protein product. The boundaries of the coding sequence are generally determined by an open reading frame (hereinafter, “ORF”), which usually begins with an ATG start codon. The coding sequence typically includes DNA, cDNA, and recombinant nucleotide sequences.

As defined herein, the term “open reading frame” (hereinafter, “ORF”) means a nucleic acid or nucleic acid sequence (whether naturally occurring, non-naturally occurring, or synthetic) comprising an uninterrupted reading frame consisting of (i) an initiation codon, (ii) a series of codons representing amino acids and (iii) a termination codon, the ORF being read (or translated) in the 5′ to 3′ direction.

The term “promoter” as used herein refers to a nucleic acid sequence capable of controlling the expression of a coding sequence or functional RNA. In general, a coding sequence is located 3′ (downstream) to a promoter sequence. Promoters may be derived in their entirety from a native gene, or be composed of different elements derived from different promoters found in nature, or even comprise synthetic nucleic acid segments. It is understood by those skilled in the art that different promoters may direct the expression of a gene in different cell types, or at different stages of development, or in response to different environmental or physiological conditions. Promoters which cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity.

The term “operably linked” as used herein refers to the association of nucleic acid sequences on a single nucleic acid fragment so that the function of one is affected by the other. For example, a promoter is operably linked with a coding sequence (e.g., an ORF) when it is capable of affecting the expression of that coding sequence (i.e., that the coding sequence is under the transcriptional control of the promoter). Coding sequences can be operably linked to regulatory sequences in sense or antisense orientation.

A nucleic acid is “operably linked” when it is placed into a functional relationship with another nucleic acid sequence. For example, DNA encoding a secretory leader (i.e., a signal peptide), is operably linked to DNA for a polypeptide if it is expressed as a pre-protein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

As used herein, “a functional promoter sequence controlling the expression of a gene of interest (or open reading frame thereof) linked to the gene of interest's protein coding sequence” refers to a promoter sequence which controls the transcription and translation of the coding sequence in a host cell of the disclosure. For example, in certain embodiments, the present disclosure is directed to a polynucleotide comprising a 5′ promoter (or 5′ promoter region, or tandem 5′ promoters and the like), wherein the promoter region is operably linked to a nucleic acid sequence encoding a protein of interest. Thus, in certain embodiments, a functional promoter sequence controls the expression of a gene of interest encoding a protein of interest.

As defined herein, “suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, RNA processing site, effector binding site and stem-loop structure.

As defined herein, the term “introducing”, as used in phrases such as “introducing into a host cell” at least one polynucleotide open reading frame (ORF), or a gene thereof, or a vector thereof, includes methods known in the art for introducing polynucleotides into a cell, including, but not limited to protoplast fusion, natural or artificial transformation (e.g., calcium chloride, electroporation), transduction, transfection, conjugation and the like (e.g., see Ferrari et al., 1989).

As used herein, “transformed” or “transformation” mean a cell has been transformed by use of recombinant DNA techniques. Transformation typically occurs by insertion of one or more nucleotide sequences (e.g., a polynucleotide, an ORF or gene) into a cell. The inserted nucleotide sequence may be a heterologous nucleotide sequence (i.e., a sequence that is not naturally occurring in cell that is to be transformed). As used herein, “transformation” refers to introducing an exogenous DNA into a host cell so that the DNA is maintained as a chromosomal integrant or a self-replicating extra-chromosomal vector.

As used herein, “transforming DNA”, “transforming sequence”, and “DNA construct” refer to DNA that is used to introduce sequences into a host cell. Transforming DNA is DNA used to introduce sequences into a host cell. The DNA may be generated in vitro by PCR or any other suitable techniques. In some embodiments, the transforming DNA comprises an incoming sequence, while in other embodiments it further comprises an incoming sequence flanked by homology boxes. In yet a further embodiment, the transforming DNA comprises other non-homologous sequences, added to the ends (i.e., stuffer sequences or flanks). The ends can be closed such that the transforming DNA forms a closed circle, such as, for example, insertion into a vector.

As used herein “an incoming sequence” refers to a DNA sequence that is introduced into the host cell chromosome. In some embodiments, the incoming sequence is part of a DNA construct. In other embodiments, the incoming sequence encodes one or more proteins of interest. In some embodiments, the incoming sequence encodes one or more proteins of interest, a gene, and/or a mutated or modified gene. In alternative embodiments, the incoming sequence encodes a functional wild-type gene or operon, a functional mutant gene or operon, or a nonfunctional gene or operon. In some embodiments, the nonfunctional sequence may be inserted into a gene to disrupt function of the gene. In another embodiment, the incoming sequence includes a selective marker. In a further embodiment the incoming sequence includes two homology boxes (e.g., up-stream and down-stream homology arms).

As used herein, “homology box” refers to a nucleic acid sequence, which is homologous to a sequence in the Bacillus chromosome. More specifically, a homology box is an upstream or downstream region having between about 80 and 100% sequence identity, between about 90 and 100% sequence identity, or between about 95 and 100% sequence identity with the immediate flanking coding region of a gene or part of a gene to be deleted, disrupted, inactivated, down-regulated and the like, according to the invention. These sequences direct where in the Bacillus chromosome a DNA construct is integrated and directs what part of the Bacillus chromosome is replaced by the incoming sequence. While not meant to limit the present disclosure, a homology box may include about between 1 base pair (bp) to 200 kilobases (kb). Preferably, a homology box includes about between 1 bp and 10.0 kb; between 1 bp and 5.0 kb; between 1 bp and 2.5 kb; between 1 bp and 1.0 kb, and between 0.25 kb and 2.5 kb. A homology box may also include about 10.0 kb, 5.0 kb, 2.5 kb, 2.0 kb, 1.5 kb, 1.0 kb, 0.5 kb, 0.25 kb and 0.1 kb. In some embodiments, the 5′ and 3′ ends of a selective marker are flanked by a homology box (homology arms) wherein the homology box comprises nucleic acid sequences immediately flanking the coding region of the gene.

As used herein, the term “selectable marker-encoding nucleotide sequence” refers to a nucleotide sequence which is capable of expression in the host cells and where expression of the selectable marker confers to cells containing the expressed gene the ability to grow in the presence of a corresponding selective agent or lack of an essential nutrient.

As used herein, the terms “selectable marker” and “selective marker” refer to a nucleic acid (e.g., a gene) capable of expression in host cell which allows for ease of selection of those hosts containing the vector. Examples of such selectable markers include, but are not limited to, antimicrobials. Thus, the term “selectable marker” refers to genes that provide an indication that a host cell has taken up an incoming DNA of interest or some other reaction has occurred. Typically, selectable markers are genes that confer antimicrobial resistance or a metabolic advantage on the host cell to allow cells containing the exogenous DNA to be distinguished from cells that have not received any exogenous sequence during the transformation.

A “residing selectable marker” is one that is located on the chromosome of the microorganism to be transformed. A residing selectable marker encodes a gene that is different from the selectable marker on the transforming DNA construct. Selective markers are well known to those of skill in the art. As indicated above, the marker can be an antimicrobial resistance marker (e.g., ampR, phleoR, specR, kanR, eryR, tetR, cmpR and neoR (e.g., see Guerot-Fleury, 1995; Palmeros et al., 2000; and Trieu-Cuot et al., 1983). In some embodiments, the present invention provides a chloramphenicol resistance gene (e.g., the gene present on pC194, as well as the resistance gene present in the Bacillus licheniformis genome). This resistance gene is particularly useful in the present invention, as well as in embodiments involving chromosomal amplification of chromosomally integrated cassettes and integrative plasmids (e.g., see Albertini and Galizzi, 1985; Stahl and Ferrari, 1984). Other markers useful in accordance with the invention include, but are not limited to auxotrophic markers, such as serine, lysine, tryptophan; and detection markers, such as β-galactosidase.

As defined herein, a host cell “genome” or a parental cell “genome” includes chromosomal and extrachromosomal genes.

As used herein, the terms “plasmid”, “vector” and “cassette” refer to extrachromosomal elements, often carrying genes which are typically not part of the central metabolism of the cell, and usually in the form of circular double-stranded DNA molecules. Such elements may be autonomously replicating sequences, genome integrating sequences, phage or nucleotide sequences, linear or circular, of a single-stranded or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

A used herein, a “transformation cassette” refers to a specific vector comprising a gene (or ORF thereof), and having elements in addition to the foreign gene that facilitate transformation of a particular host cell.

As used herein, the term “vector” refers to any nucleic acid that can be replicated (propagated) in cells and can carry new genes or DNA segments into cells. Thus, the term refers to a nucleic acid construct designed for transfer between different host cells. Vectors include viruses, bacteriophage, pro-viruses, plasmids, phagemids, transposons, and artificial chromosomes such as YACs (yeast artificial chromosomes), BACs (bacterial artificial chromosomes), PLACs (plant artificial chromosomes), and the like, that are “episomes” (i.e., replicate autonomously or can integrate into a chromosome of a host organism).

An “expression vector” refers to a vector that has the ability to incorporate and express heterologous DNA in a cell. Many prokaryotic and eukaryotic expression vectors are commercially available and know to one skilled in the art. Selection of appropriate expression vectors is within the knowledge of one skilled in the art.

As used herein, the terms “expression cassette” and “expression vector” refer to a nucleic acid construct generated recombinantly or synthetically, with a series of specified nucleic acid elements that permit transcription of a particular nucleic acid in a target cell (i.e., these are vectors or vector elements, as described above). The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter. In some embodiments, DNA constructs also include a series of specified nucleic acid elements that permit transcription of a particular nucleic acid in a target cell. In certain embodiments, a DNA construct of the disclosure comprises a selective marker and an inactivating chromosomal or gene or DNA segment as defined herein.

As used herein, a “targeting vector” is a vector that includes polynucleotide sequences that are homologous to a region in the chromosome of a host cell into which the targeting vector is transformed and that can drive homologous recombination at that region. For example, targeting vectors find use in introducing mutations into the chromosome of a host cell through homologous recombination. In some embodiments, the targeting vector comprises other non-homologous sequences, e.g., added to the ends (i.e., stuffer sequences or flanking sequences). The ends can be closed such that the targeting vector forms a closed circle, such as, for example, insertion into a vector. Selection and/or construction of appropriate vectors is well within the knowledge of those having skill in the art.

As used herein, the term “plasmid” refers to a circular double-stranded (ds) DNA construct used as a cloning vector, and which forms an extrachromosomal self-replicating genetic element in many bacteria and some eukaryotes. In some embodiments, plasmids become incorporated into the genome of the host cell.

In certain embodiments, a host cell of the disclosure expresses/produces an increased amount of a CAH and/or an increased amount of a BH. In certain embodiments, an increased amount of the CAH expressed/produced by the host cell is at least a 0.05% increase, at least 0.10%, at least a 1.0% increase, at least a 5.0% increase, or a greater than 5.0% increase. In other embodiments, an increased amount of the BH expressed/produced by the host cell is at least a 0.05% increase, at least 0.10%, at least a 1.0% increase, at least a 5.0% increase, or a greater than 5.0% increase. As a non-limiting example, in certain embodiments, an increased amount of a cyanuric acid hydrolase and/or a biuret hydrolase produced is detected or measured as an increase in enzymatic activity and/or an increase specific productivity (Qp).

As used herein, the terms “polypeptide” and “protein” are used interchangeably, and refer to polymers of any length comprising amino acid residues linked by peptide bonds. The conventional one (1) letter or three (3) letter codes for amino acid residues are used herein. The polypeptide may be linear or branched, it may comprise modified amino acids, and it may be interrupted by non-amino acids. The term polypeptide also encompasses an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

As used herein, a “variant” polypeptide refers to a polypeptide that is derived from a parent (or reference) polypeptide by the substitution, addition, or deletion of one or more amino acids, typically by recombinant DNA techniques. Variant polypeptides may differ from a parent polypeptide by a small number of amino acid residues and may be defined by their level of primary amino acid sequence homology/identity with a parent (reference) polypeptide.

In certain embodiments, variant polypeptides have at least 40%, at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, or even at least 99% amino acid sequence identity with a parent (reference) polypeptide sequence. As used herein, a “variant” polynucleotide refers to a polynucleotide encoding a variant polypeptide, wherein the “variant polynucleotide” has a specified degree of sequence homology/identity with a parent polynucleotide, or hybridizes with a parent polynucleotide (or a complement thereof) under stringent hybridization conditions. In certain embodiments, a variant polynucleotide has at least 70%, at least 75%, at least 80%, at least 85%, at least 90%, at least 91%, at least 92%, at least 93%, at least 94%, at least 95%, at least 96%, at least 97%, at least 98%, or even at least 99% nucleotide sequence identity with a parent (reference) polynucleotide sequence.

As used herein, a “mutation” refers to any change or alteration in a nucleic acid sequence. Several types of mutations exist, including point mutations, deletion mutations, silent mutations, frame shift mutations, splicing mutations and the like. Mutations may be performed specifically (e.g., via site directed mutagenesis) or randomly (e.g., via chemical agents, passage through repair minus bacterial strains).

As used herein, in the context of a polypeptide or a sequence thereof, the term “substitution” means the replacement (i.e., substitution) of one amino acid with another amino acid.

As defined herein, a “heterologous control sequence”, refers to a gene expression control sequence (e.g., a promoter or enhancer) which does not function in nature to regulate (control) the expression of the gene of interest. Generally, heterologous nucleic acid sequences are not endogenous (native) to the cell, or a part of the genome in which they are present, and have been added to the cell, by infection, transfection, transformation, microinjection, electroporation, and the like. A “heterologous” nucleic acid construct may contain a control sequence/DNA coding (ORF) sequence combination that is the same as, or different, from a control sequence/DNA coding sequence combination found in the native host cell.

As used herein, the terms “signal sequence” and “signal peptide” refer to a sequence of amino acid residues that may participate in the secretion or direct transport of a mature protein or precursor form of a protein. The signal sequence is typically located N-terminal to the precursor or mature protein sequence. The signal sequence may be endogenous or exogenous. A signal sequence is normally absent from the mature protein. A signal sequence is typically cleaved from the protein by a signal peptidase after the protein is transported.

The term “derived” encompasses the terms “originated” “obtained,” “obtainable,” and “created,” and generally indicates that one specified material or composition finds its origin in another specified material or composition, or has features that can be described with reference to the another specified material or composition.

As used herein, the term “homology” relates to homologous polynucleotides or polypeptides. If two or more polynucleotides or two or more polypeptides are homologous, this means that the homologous polynucleotides or polypeptides have a “degree of identity” of at least 45%, 50%, 60%, 70%, 85%, 95%, 98% and the like. Whether two polynucleotide or polypeptide sequences have a sufficiently high degree of identity to be homologous as defined herein, can suitably be investigated by aligning the two sequences using a computer program known in the art, such as “GAP” provided in the GCG program package (Program Manual for the Wisconsin Package, Version 8, August 1994, Genetics Computer Group, 575 Science Drive, Madison, Wis., USA 53711) (Needleman and Wunsch, (1970). Using GAP with the following settings for DNA sequence comparison: GAP creation penalty of 5.0 and GAP extension penalty of 0.3.

As used herein, the term “percent (%) identity” refers to the level of nucleic acid or amino acid sequence identity between the nucleic acid sequences that encode a polypeptide or the polypeptide's amino acid sequences, when aligned using a sequence alignment program.

As used herein, “specific productivity” is total amount of protein produced per cell per time over a given time period.

As defined herein, the terms “purified”, “isolated” or “enriched” are meant that a biomolecule (e.g., a polypeptide or polynucleotide) is altered from its natural state by virtue of separating it from some, or all of, the naturally occurring constituents with which it is associated in nature. Such isolation or purification may be accomplished by art-recognized separation techniques such as ion exchange chromatography, affinity chromatography, hydrophobic separation, dialysis, protease treatment, ammonium sulphate precipitation or other protein salt precipitation, centrifugation, size exclusion chromatography, filtration, microfiltration, gel electrophoresis or separation on a gradient to remove whole cells, cell debris, impurities, extraneous proteins, or enzymes undesired in the final composition. It is further possible to then add constituents to a purified or isolated biomolecule composition which provide additional benefits, for example, activating agents, anti-inhibition agents, desirable ions, compounds to control pH or other enzymes or chemicals.

As used herein, “homologous genes” refers to a pair of genes from different, but usually related species, which correspond to each other and which are identical or very similar to each other. The term encompasses genes that are separated by speciation (i.e., the development of new species) (e.g., orthologous genes), as well as genes that have been separated by genetic duplication (e.g., paralogous genes).

As used herein, a “flanking sequence” refers to any sequence that is either upstream or downstream of the sequence being discussed (e.g., for genes A-B-C, gene B is flanked by the A and C gene sequences). In certain embodiments, the incoming sequence is flanked by a homology box on each side. In another embodiment, the incoming sequence and the homology boxes comprise a unit that is flanked by stutter sequence on each side. In some embodiments, a flanking sequence is present on only a single side (either 3′ or 5′), but in preferred embodiments, it is on each side of the sequence being flanked. The sequence of each homology box is homologous to a sequence in the Bacillus chromosome. These sequences direct where in the Bacillus chromosome the new construct gets integrated and what part of the Bacillus chromosome will be replaced by the incoming sequence. In other embodiments, the 5′ and 3′ ends of a selective marker are flanked by a polynucleotide sequence comprising a section of the inactivating chromosomal segment. In some embodiments, a flanking sequence is present on only a single side (either 3′ or 5′), while in other embodiments, it is present on each side of the sequence being flanked.

### II. Parental Cells Comprising Endogenous CAH Genes or Endogenous Biuret Hydrolase Genes

As generally set forth above, certain embodiments of the disclosure are related to the heterologous expression/production of CAH enzymes and/or BH enzymes in one or more microbial host cells of the disclosure. In certain embodiments, the disclosure is therefore related to native genes encoding such CAH enzymes and/or BH enzymes for the heterologous expression/production in a microbial host cell.

In certain embodiments, a gene (or ORF) encoding a cyanuric acid hydrolase (CAH) is derived from a parental cell comprising an endogenous CAH gene. In certain embodiments, a parental cell comprising an endogenous gene encoding a CAH is selected from the group consisting of a M. thermoacetica cell, a B. diazoefficiens cell, a Bradyrhizobium sp. (WSM1253) cell, a Pseudolabrys sp. (Root1462) cell and an A. citrulli (122227) cell.

In other embodiments, a gene (or ORF) of the disclosure encodes a CAH comprising at least about 45% amino acid sequence identity to a CAH enzyme selected from the group consisting of SEQ ID NO: 2, SEQ ID NO: 4, SEQ ID NO: 6, SEQ ID NO: 8, SEQ ID NO: 10 and SEQ ID NO: 12. For example, Table 1 below lists exemplary enzymes comprising CAH activity and their relative amino acid sequence identity.

TABLE 1
CYANURIC ACID HYDROLASES AND THEIR RELATIVE
AMINO ACID SEQUENCE IDENTITY

| | Parental Cell | % ID to | % ID to | % ID to | % ID to | % ID to | % ID to |
|---|---|---|---|---|---|---|---|
| SID | (Source of CAH) | SID 2 | SID 4 | SID 6 | SID 8 | SID 10 | SID 12 |
| 2 | M. thermoacetica (WT) | 100 | 99.7 | 58.3 | 56.9 | 51 | 63.4 |
| 4 | M. thermoacetica | 99.7 | 100 | 58.3 | 56.9 | 51 | 63.1 |
| | (C46A) | | | | | | |
| 6 | Bradyrhizobium | 58.3 | 58.3 | 100 | 89.5 | 51.4 | 55.4 |
| | diazoefficiens | | | | | | |
| 8 | Bradyrhyzobium sp. | 56.9 | 56.9 | 89.5 | 100 | 50.4 | 55.4 |
| | (WSM1253) | | | | | | |
| 10 | Pseudolabrys sp. | 51 | 51 | 51.4 | 50.5 | 100 | 49.5 |
| | (Root1462) | | | | | | |
| 12 | Acidovorax citrulli | 63.4 | 63.1 | 55.4 | 55.4 | 49.5 | 100 |
| | (122227) | | | | | | |

The term “sequence identity number” or “SEQ ID NO” has been abbreviated as “SID” in Table 1.

In other embodiments, a gene (or ORF) of the disclosure encodes a protein comprising CAH activity, wherein the encoded protein comprises a consensus sequence set forth in SEQ ID NO: 23.

In other embodiments, a gene (or ORF) encoding a biuret hydrolase (BH) is derived from a parental cell comprising an endogenous BH gene. In certain embodiments, a parental cell comprising an endogenous gene encoding a BH is a Herbaspirillum sp. (BH-1) cell. In other embodiments, a gene (or ORF) of the disclosure encodes a BH comprising at least about 50% amino acid sequence identity to a BH enzyme of SEQ ID NO: 14.

### III. Microbial Host Cells for the Enhanced Production of Heterologous CAH and BH

Thus, as generally described above, certain embodiments of the disclosure are related to the heterologous expression/production of CAH enzymes and/or BH enzymes in one or more microbial host cells of the disclosure. As generally set forth above in Section II, various parental cells comprise endogenous genes encoding CAH or BH, and as such, the CAH or BH genes in these parental cells may be cloned, synthesized and the like, for the heterologous expression of the same in a microbial host cell of the disclosure. As generally described in the following sections, Applicant constructed and optimized exemplary microbial host cells for the expression of heterologous CAH genes (ORFs) and BH genes (ORFs). Thus, construction and optimization of the microbial host cells as described herein are particularly well-suited for growth in submerged cultures for the large-scale production of heterologous CAH and BH proteins, e.g., for use in CYA remediation processes.

A. Bacillus sp. Host Cells

Thus, in certain embodiments, a microbial host cell of the disclosure is a Bacillus sp. host cell. For example, PCT Publication No. WO2002/14490 (incorporated herein by reference in its entirety) discloses methods for modifying Bacillus sp. cells including (1) the construction and transformation of an integrative plasmid (pComK), (2) random mutagenesis of coding sequences, signal sequences and pro-peptide sequences, (3) homologous recombination, (4) increasing transformation efficiency by adding non-homologous flanks to the transformation DNA, (5) optimizing double cross-over integrations, (6) site directed mutagenesis and (7) marker-less deletion. PCT Publication No. WO2003/083125 further discloses methods for modifying Bacillus sp. cells, such as the creation of Bacillus deletion strains and DNA constructs using PCR fusion to bypass E. coli.

Those of skill in the art are well aware of suitable methods for introducing polynucleotide sequences into bacterial cells (e.g., E. coli and Bacillus sp.) (e.g., Ferrari et al., 1989; Saunders et al., 1984; Hoch et al., 1967; Mann et al., 1986; Holubova, 1985; Chang et al., 1979; Vorobjeva et al., 1980; Smith et al., 1986; Fisher et. al., 1981 and McDonald, 1984). Indeed, such methods as transformation including protoplast transformation and congression, transduction, and protoplast fusion are known and suited for use in the present disclosure. Methods of transformation are particularly preferred to introduce a DNA construct of the present disclosure into a Bacillus sp. host cell.

In addition to commonly used methods, in some embodiments, host cells are directly transformed (i.e., an intermediate cell is not used to amplify, or otherwise process, the DNA construct prior to introduction into the host cell). Introduction of the DNA construct into the host cell includes those physical and chemical methods known in the art to introduce DNA into a host cell, without insertion into a plasmid or vector. Such methods include, but are not limited to, calcium chloride precipitation, electroporation, naked DNA, liposomes and the like. In additional embodiments, DNA constructs are co-transformed with a plasmid without being inserted into the plasmid. In further embodiments, a selective marker is deleted or substantially excised from the modified Bacillus strain by methods known in the art (e.g., Stahl et al., 1984 and Palmeros et al., 2000). In some embodiments, resolution of the vector from a host chromosome leaves the flanking regions in the chromosome, while removing the indigenous chromosomal region.

Promoters and promoter sequence regions for use in the expression of genes, open reading frames (ORFs) thereof and/or variant sequences thereof in Bacillus sp. cells are generally known on one of skill in the art. Promoter sequences of the disclosure of the disclosure are generally chosen so that they are functional in the Bacillus sp. cells. For example, promoters useful for driving gene expression in Bacillus sp. cells include, but are not limited to, the B. subtilis alkaline protease (aprE) promoter (Stahl et al., 1984), the α-amylase promoter of B. subtilis (Yang et al., 1983), the α-amylase promoter of B. amyloliquefaciens (Tarkinen et al., 1983), the neutral protease (nprE) promoter from B. subtilis (Yang et al., 1984), a mutant aprE promoter (PCT Publication No. WO2001/51643), a ribosomal protein promoter or a ribosomal RNA promoter (e.g., the rrnl promoter described in U.S. Patent Publication No. 2014/0329309) and the like. Methods for screening and creating promoter libraries with a range of activities (promoter strength) in Bacillus cells is describe in PCT Publication No. WO2003/089604.

B. Filamentous Fungal Host Cells

In certain embodiments, a microbial host cell of the disclosure is a filamentous fungal cell. The construction and manipulation of filamentous fungal cells (e.g., Trichoderma sp. cells, Aspergillus sp. cells and the like) for the recombinant productions of proteins of interest have been described in the art (e.g., see PCT Publication Nos. WO2011/075677, WO2016/130523, WO2017/019867 and WO2018/093752, each incorporated herein by reference). In certain embodiments filamentous fungal cells are constructed by introducing, substituting, and/or removing one or more (several) nucleotides in a gene or a control sequence thereof required for the transcription or translation thereof. Such modifications may be accomplished by site-directed mutagenesis or PCR generated mutagenesis in accordance with methods known in the art (e.g., Botstein and Shortle, 1985; Lo et al., 1985; Higuchi et al., 1988; Shimada, 1996; Ho et al., 1989; Horton et al., 1989 and Sarkar and Sommer, 1990). In certain embodiments, filamentous fungal cells are constructed (genetically modified) by means of CRISPR/Cas9 editing. More specifically, compositions and methods for fungal genome modification by CRISPR/Cas9 systems are described and well known in the art (e.g., see, International PCT Publication Nos: WO2016/100571, WO2016/100568, WO2016/100272, WO2016/100562 and the like).

Thus, as described herein, an isolated polynucleotide encoding a heterologous CAH or BH protein may be manipulated in a variety of ways to provide for expression of the polypeptide in a filamentous fungal host cell of the disclosure. Manipulation of the polynucleotide's sequence prior to its insertion into a vector may be desirable or necessary depending on the expression vector. The techniques for modifying polynucleotide sequences utilizing recombinant DNA methods are well known in the art. A nucleic acid construct comprising a polynucleotide encoding a heterologous CAH or BH polypeptide may be operably linked to one or more (several) control sequences capable of directing expression of the coding sequence in a filamentous fungal cell of the disclosure, under conditions compatible with the control sequences.

The control sequence may be an appropriate promoter sequence, a nucleotide sequence that is recognized by filamentous fungal cell of the disclosure for expression of the polynucleotide encoding the polypeptide. The promoter sequence contains transcriptional control sequences that mediate expression of the CAH or BH polypeptide. The promoter may be any nucleotide sequence that shows transcriptional activity in the fungal host cell, including mutant, truncated, and hybrid promoters, and may be obtained from genes encoding extracellular or intracellular polypeptides either native or heterologous (foreign) to the filamentous fungal (host) cell.

Examples of suitable promoters for directing the transcription of the nucleic acid constructs in the methods of the present disclosure are promoters obtained or derived from the genes for Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, Fusarium venenatum amyloglucosidase, Fusarium venenatum Dana, Fusarium venenatum Quinn, Fusarium oxysporum trypsin-like protease, Trichoderma reesei beta-glucosidase, Trichoderma reesei cellobiohydrolase I (cbhI), T. reesei cellobiohydrolase II (cbhII), T. reesei endoglucanase I (egI), T. reesei endoglucanase II (egII), T. reesei endoglucanase III (egIII), T. reesei endoglucanase V (egV), T. reesei xylanase I (xyl1), T. reesei xylanase II (xyl2), T. reesei beta-xylosidase and the like, including mutant, truncated, and hybrid promoters thereof.

The control sequence may also be a suitable transcription terminator sequence, a sequence recognized by a filamentous fungal host cell of the disclosure to terminate transcription. The terminator sequence is operably linked to the 3′ terminus of the nucleotide sequence encoding the heterologous CAH or BH polypeptide. Any terminator that is functional in a filamentous fungal host cell of the disclosure may be used. Exemplary terminators may be obtained or derived from the genes for Aspergillus oryzae TAKA amylase, Aspergillus niger glucoamylase, Aspergillus nidulans anthranilate synthase, Aspergillus niger alpha-glucosidase and Fusarium oxysporum trypsin-like protease.

The control sequence may also be a suitable leader sequence, a non-translated region of a mRNA that is important for translation by a fungal host cell of the disclosure. The leader sequence is operably linked to the 5′ terminus of the nucleotide sequence encoding the heterologous CAH or BH polypeptide. Any leader sequence that is functional in a filamentous fungal host cell of the disclosure may be used in the present invention. The control sequence may also be a polyadenylation sequence, a sequence operably linked to the 3′ terminus of the nucleotide sequence and, when transcribed, is recognized by filamentous fungal host cell as a signal to add polyadenosine residues to transcribed mRNA. Any polyadenylation sequence that is functional in filamentous fungal host cell of the disclosure may be used.

The control sequence may also be a signal peptide coding sequence that codes for an amino acid sequence linked to the amino terminus of a polypeptide and directs the encoded polypeptide into the cell's secretory pathway. The 5′ end of the coding sequence of the nucleotide sequence may inherently contain a signal peptide coding sequence naturally linked in translation reading frame with the segment of the coding sequence that encodes the secreted polypeptide. Alternatively, the 5′ end of the coding sequence may contain a signal peptide coding sequence that is foreign to the coding sequence. The foreign signal peptide coding sequence may be required where the coding sequence does not naturally contain a signal peptide coding sequence. Alternatively, the foreign signal peptide coding sequence may simply replace the natural signal peptide coding sequence in order to enhance secretion of the polypeptide. However, any signal peptide coding sequence that directs the expressed CAH or BH polypeptide into the secretory pathway of a filamentous fungal host cell of the disclosure, i.e., secreted into a culture medium may be used.

Thus, in certain embodiments, a recombinant expression vector comprising a nucleotide sequence, a promoter, and transcriptional and translational stop signals may be used for the recombinant production of a heterologous CAH or BH polypeptide. For example, the various nucleic acids and control sequences described herein may be joined together to produce a recombinant expression vector that may include one or more (several) convenient restriction sites to allow for insertion or substitution of the nucleotide sequence encoding the CAH or BH polypeptide at such sites. Alternatively, the nucleotide sequence may be expressed by inserting the nucleotide sequence or a nucleic acid construct comprising the sequence into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the nucleotide sequence. The choice of the vector will typically depend on its compatibility with the filamentous fungal host cell into which the vector is to be introduced. The vector may be a linear or closed circular plasmid. The vector may be an autonomously replicating vector (i.e., a vector that exists as an extrachromosomal entity, the replication of which is independent of chromosomal replication), e.g., a plasmid, an extrachromosomal element, a mini-chromosome, or an artificial chromosome. The vector may contain any means for assuring self-replication. Alternatively, the vector may be one that, when introduced into a filamentous fungal host cell, is integrated into the genome and replicated together with the chromosome(s) into which it has been integrated. Furthermore, a single vector or plasmid may be used, or two or more vectors or plasmids maybe used, that together contain the total DNA to be introduced into the genome of the host cell or a transposon. In certain embodiments, the vector contains one or more (several) selectable markers that permit easy selection of transformed host cell. Examples of selectable markers for use in a filamentous fungal host cell of the disclosure include, but are not limited to, amdS (acetamidase), argB (ornithine carbamoyltransferase), bar (phosphinothricin acetyltransferase), hpt (hygromycin phosphotransferase), niaD (nitrate reductase), pyrG (orotidine-5′-phosphate decarboxylase), sC (sulfate adenyltransferase) and trpC (anthranilate synthase), as well as equivalents thereof.

The vectors preferably contain an element(s) that permits integration of the vector into the host cell's genome or autonomous replication of the vector in the cell independent of the genome. For integration into the genome of a fungal host cell, the vector may rely on the polynucleotide's sequence encoding the polypeptide or any other element of the vector for integration into the genome by homologous or non-homologous recombination. Alternatively, the vector may contain additional nucleotide sequences for directing integration by homologous recombination into the genome of fungal host at a precise location(s) in the chromosome(s). The integrational elements may be any sequence that is homologous with the target sequence in the genome of filamentous fungal host cell. Furthermore, the integrational elements may be non-encoding or encoding nucleotide sequences. On the other hand, the vector may be integrated into the genome of the host cell by non-homologous recombination. For autonomous replication, the vector may further comprise an origin of replication enabling the vector to replicate autonomously in the fungal host cell. The origin of replication may be any plasmid replicator mediating autonomous replication that functions in a cell. Thus, the procedures used to ligate the elements described herein to construct recombinant expression vectors are well known to one skilled in the art (e.g., see Molecular Cloning, A Laboratory Manual, 2^{nd} edition, 1989). In certain embodiments, the introduction of an expression vector into a fungal host cell may involve a process consisting of protoplast formation, transformation of the protoplasts, and regeneration of the strain wall in a manner known per se. For example, suitable procedures for transformation of Trichoderma sp. strains are described in Malardier et al. (1989) and PCT Publication No. WO1996/00787.

In certain embodiments, filamentous fungal cells are cultivated in a nutrient medium for production of the CAH and/or BH protein using methods known in the art. For example, the host cell may be cultivated by shake flask cultivation, small-scale or large-scale fermentation (including continuous, batch, fed-batch, or solid state fermentations) in laboratory or industrial fermentors performed in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). The secreted polypeptide can be recovered directly from the medium. If the polypeptide is not secreted, it may be obtained from cell lysates.

In certain embodiments, a CAH and/or BH polypeptide is detected using methods known in the art that are specific for the polypeptide. These detection methods may include use of specific antibodies, high performance liquid chromatography, capillary chromatography, formation of an enzyme product, disappearance of an enzyme substrate, or SDS-PAGE. For example, an enzyme assay may be used to determine the activity of a CAH or BH enzyme.

The resulting CAH or BH polypeptide is isolated by methods known in the art. For example, a CAH or BH polypeptide of interest may be isolated from the cultivation medium by conventional procedures including, but not limited to, centrifugation, filtration, extraction, spray-drying, evaporation, or precipitation. The isolated polypeptide may then be further purified by a variety of procedures known in the art including, but not limited to, chromatography (e.g., ion exchange, affinity, hydrophobic, chromatofocusing, and size exclusion), electrophoretic procedures (e.g., preparative isoelectric focusing (IEF), differential solubility (e.g., ammonium sulfate precipitation) or extraction (e.g., see, Protein Purification, 1989).

C. Yeast Host Cells

In certain embodiments, a microbial host cell of the disclosure is a yeast cell. The construction and manipulation of yeast cells (e.g., Saccharomyces cerevisiae and the like) for the recombinant productions of proteins of interest have been described in the art (e.g., see Petes et al., 1989; Ostergaard et al., 2000; Nevoigt, 2008). In certain embodiments yeast cells are constructed by introducing a heterologous gene (ORF) encoding a CAH or a BH into yeast cells by any appropriate means used in the art. For example, suitable transformation techniques are well described in the literature.

Exemplary parental cells comprising an endogenous gene encoding a CAH, or comprising an endogenous gene encoding a BH, are generally described and set forth above in Section II. In the instant example, Applicant evaluated the heterologous expression of genes (SEQ ID NO: 5 and SEQ ID NO: 9) encoding CAH homologue of SEQ ID NO: 6 and SEQ ID NO: 10, respectively. More particularly, the present example describes the construction and optimization Saccharomyces cerevisiae host cells (an exemplary yeast cell) for the heterologous expression of two CAH (enzyme) homologues. However, as described herein, the S. cerevisiae cell is not meant to limit the choice of a host cell, as any other yeast cell (or any other microbial host cell of the disclosure) may be adapted and used herein for the heterologous expression of a gene encoding a CAH.

The nucleic acid molecules (polynucleotides) described herein are operatively linked to expression control sequences, recombinant DNA cloning vectors, vectors containing such a recombinant DNA molecule, and the like. Alternatively, the naked nucleic acid molecule (polynucleotide) may be introduced directly into the cell. Conveniently, transformation of yeast cells as described herein is effected by the lithium acetate transformation method (Burke et al., 2000), by freeze-thaw methods, spheroplast methods or other appropriate techniques (e.g., see, Dohmen et al., 1991). Expression vectors are described herein include appropriate control sequences, such as for example translational (e.g. start and stop codons, ribosomal binding sites) and transcriptional control elements (e.g. promoter-operator regions, termination stop sequences) operably linked in matching reading frame with a nucleic acid molecule encoding a CAH or BH homologue. Suitable vectors include plasmids and viruses (including both bacteriophage and eukaryotic viruses), wherein suitable viral vectors include baculovirus adenovirus, adeno-associated virus, herpes and vaccinia/pox viruses, in addition to other viral vectors are known in the art.

In certain embodiments, a yeast cell of the disclosure is cultivated/fermented under suitable conditions for the expression/production of the encoded CAH or BH homologue. For example, culturing may be performed in a culture medium including a carbon source (e.g., glucose). The medium used in yeast cell culturing may be any general medium appropriate for growth of a host cell, such as a minimal medium or a complex medium including an appropriate supplement. The medium used in the culturing may be a medium capable of satisfying specific yeast cell requirements. For example, the medium may be a medium such as a carbon source, a nitrogen source, a salt, a trace element, and a combination thereof. In certain embodiments, a carbon source which may be used by a yeast cell includes, but is not limited to, monosaccharides, disaccharides and polysaccharides. The carbon source may be an assimilable sugar, such as a hexose or a pentose. Specifically, glucose, fructose, mannose, galactose or others may be used as the carbon source. A nitrogen source which may be used by a yeast cell is an organic nitrogen compound, or an inorganic nitrogen compound. Oxygen conditions for culturing a yeast cell may be aerobic conditions having a normal oxygen partial pressure, low-oxygen conditions including oxygen from about 0.1% to about 10%, for example, from about 0.1% to about 8%, from about 0.1% to about 6%, from about 0.1% to about 4%, from about 0.1% to about 2%, from about 0.1% to about 1%, from about 1% to about 10%, from about 1% to about 8%, from about 1% to about 6%, from about 2% to about 10%, from about 4% to about 10%, from about 6% to about 10%, from about 8% to about 10%, from about 2% to about 8%, or from about 2% to about 6% in the atmosphere, or anaerobic conditions including no oxygen.

The CAH or BH expressed/produced by the yeast host cell is recovered or isolated by methods known in the art, such as centrifugation, filtration, ion-exchange chromatography, crystallization and the like. For example, the culture solution (broth) may be centrifuged at a low speed to remove biomass and the resulting supernatant may be separated by ion-exchange chromatography. The recovery may be recovery from a cell, a culture medium, or from both a cell and a culture medium.

### IV. Heterologously Expressed CAH and BH Proteins

As briefly stated in the preceding sections, the present microbial host cells and methods find use in the production of commercially important CAH and BH proteins (e.g., as used in CYA remediation processes). For example, in certain embodiments, a microbial host cell of the disclosure exhibits an increased CAH or BH protein titer, wherein protein titer is defined as the amount of protein per volume (g/L). Titers can be measured by methods known in the art (e.g., ELISA, HPLC, Bradford assay, LC/MS and the like). Thus, in certain embodiments, a microbial host cell comprises a protein titer increase of at least about 0.1%, at least about 1%, at least about 5%, at least about 6%, at least about 7%, at least about 8%, at least about 9%, or at least about 10% or more, as compared to a control cell of the disclosure and/or as compared to an E. coli host cell.

In certain embodiments, a microbial host cell exhibits an increased volumetric productivity relative to a control cell of the disclosure and/or relative to an E. coli host cell, wherein volumetric productivity is defined as the amount of CAH or BH protein produced (g) during the fermentation per nominal volume (L) of the bioreactor per total fermentation time (h). For example, volumetric productivities can be measured by methods know in the art (e.g., ELISA, HPLC, Bradford assay, LC/MS and the like). Thus, in certain embodiments, a microbial host cell of the disclosure comprises a volumetric productivity increase of at least about 0.1%, at least about 1%, at least about 5%, at least about 6%, at least about 7%, at least about 8%, at least about 9%, or at least about 10% or more, relative to a control cell of the disclosure and/or relative to an E. coli host cell.

In certain other embodiments, a microbial host cell exhibits an increased total CAH or BH protein yield relative to a control cell of the disclosure and/or relative to an E. coli host cell, wherein total protein yield is defined as the amount of protein produced (g) per gram of carbohydrate fed (i.e., relative to a control cell of the disclosure and/or relative to an E. coli host cell). Thus, as used herein, total protein yield (g/g) may be calculated using the following equation:

Y_{f}=Tₚ/T_{c}

wherein “Y_{f}” is total protein yield (g/g), “Tₚ” is the total protein produced during the fermentation (g) and “T_{c}” is the total carbohydrate (g) fed during the fermentation (bioreactor) run. In certain embodiments, the increase in total protein yield of the microbial cell (i.e., relative to a control cell of the disclosure and/or relative to an E. coli host cell) is an increase of at least about 0.1%, at least about 1%, at least about 5%, at least about 6%, at least about 7%, at least about 8%, at least about 9%, or at least about 10%.

Total protein yield may also be described as carbon conversion efficiency/carbon yield, for example, as in the percentage (%) of carbon fed that is incorporated into total protein. Thus, in certain embodiments, a microbial host cell of the disclosure comprises an increased carbon conversion efficiency (e.g., an increase in the percentage (%) of carbon fed that is incorporated into total protein), relative to a control cell of the disclosure and/or relative to an E. coli host cell. In certain embodiments, the increase in carbon conversion efficiency of the microbial host cell (i.e., relative to a control cell of the disclosure and/or relative to an E. coli host cell) is an increase of at least about 0.1%, at least about 1%, at least about 5%, at least about 6%, at least about 7%, at least about 8%, at least about 9%, or at least about 10%.

In certain embodiments, a microbial host cell of the disclosure exhibits an increased specific productivity (Qp) of a CAH or BH protein, relative to a control cell of the disclosure and/or relative to an E. coli host cell. For example, the detection of specific productivity (Qp) is a suitable method for evaluating rate of protein production. The specific productivity (Qp) can be determined using the following equation:

“Qp=gP/gDCW·hr”

wherein, “gP” is grams of protein produced in the tank; “gDCW” is grams of dry cell weight (DCW) in the tank and “hr” is fermentation time in hours from the time of inoculation, which includes the time of production as well as growth time. Thus, in certain embodiments, a microbial host cell comprises a specific productivity (Qp) increase of at least about 0.1%, at least about 1%, at least about 5%, at least about 6%, at least about 7%, at least about 8%, at least about 9%, or at least about 10% or more, as compared to a control cell of the disclosure and/or an E. coli host cell.

### V. Exemplary Embodiments

Non-limiting embodiments of the disclosure include, but are not limited to:

1. A host cell comprising a heterologous polynucleotide encoding a cyanuric acid hydrolase (CAH) and/or comprising a heterologous polynucleotide encoding a heterologous biuret hydrolase (BH).

2. The host cell of embodiment 1, wherein the heterologous polynucleotide encoding the CAH is derived from a parental cell selected from the group consisting of M. thermoacetica, B. diazoefficiens, Bradyrhizobium sp. (WSM1253), Pseudolabrys sp. (Root1462) and A. citrulli (122227) and/or wherein the heterologous polynucleotide encoding the BH is derived from a Herbaspirillum sp. (BH-1) parental cell.

3. The host cell of embodiment 1, wherein the CAH encoded by the heterologous polynucleotide comprises 45% or greater amino acid sequence identity to any one of SEQ ID NO: 2, SEQ ID NO: 6, SEQ ID NO: 8, SEQ ID NO: 10 and SEQ ID NO:12.

4. The host cell of embodiment 1, wherein the CAH encoded by the heterologous polynucleotide comprises SEQ ID NO: 23.

5. The host cell of embodiment 1, wherein the CAH encoded by the heterologous polynucleotide is an EC 3.5.2.15 enzyme.

6. The host cell of embodiment 1, wherein the BH encoded by the heterologous polynucleotide is an EC 3.5.1.84 enzyme.

7. The host cell of any one of embodiments 1-6, selected from a prokaryotic host cell and a eukaryotic microbial host cell.

8. The host of any one of embodiments 1-6, wherein the prokaryotic host cell excludes Gram-negative bacterial cells.

9. The host cell of any one of embodiments 1-7, selected from the group consisting of a Gram-positive bacterial cell, a filamentous fungal cell and a yeast cell.

10. The host cell of any one of embodiments 1-9, wherein the heterologous polynucleotide encoding the CAH is a polynucleotide expression cassette and/or the heterologous polynucleotide encoding the BH is a polynucleotide expression cassette.

11. The host cell of any one of embodiments 1-10, wherein the host cell does not comprise an antimicrobial resistance gene.

12. The host cell of any one of embodiments 1-11, fermented under suitable conditions to express and produce the heterologous CAH and/or express and produce the heterologous BH.

13. The host cell of embodiment 12, wherein the heterologous CAH expressed and produced is isolated from the host cell or the host cell fermentation broth, and/or wherein the heterologous BH expressed and produced is isolated from the host cell or the host cell fermentation broth.

14. The host cell of embodiment 12, wherein the host cell is lysed after being fermented under suitable conditions to express and produce the heterologous CAH and/or heterologous BH.

15. The host cell of embodiment 14, wherein the lysed host cell fermentation broth comprises the heterologously produced CAH and/or BH.

16. The host cell of embodiment 15, where the CAH and/or BH in the lysed host cell fermentation broth is at least partially purified to remove the lysed cells from the fermentation broth.

17. The host cell embodiment 15, wherein the lysed host cell fermentation broth comprising the partially purified CAH and/or BH is substantially free of intact or viable host cell.

18. An isolated CAH and/or isolated BH produced by the host cell of embodiment 12, wherein the isolated CAH comprises no detectable lipopolysaccharides (LPS) and/or the isolated BH comprises no detectable LPS.

19. An isolated CAH produced by the host cell of embodiment 12, wherein the isolated CAH comprises equivalent or increased activity relative to the activity of same CAH endogenously produced and isolated from the parental cell from which the CAH gene was derived.

20. An isolated CAH produced by the host cell of embodiment 12, wherein the isolated CAH comprises equivalent or increased activity relative to the activity of same CAH heterologously expressed and isolated from an E. coli host cell.

21. An isolated CAH produced by the host cell of embodiment 12, wherein the isolated CAH does not comprises a poly-histidine tag.

22. An isolated BH produced by the host cell of embodiment 12, wherein the isolated BH comprises equivalent or increased activity relative to the activity of same BH endogenously produced and isolated from the parental cell from which the BH gene was derived.

23. An isolated BH produced by the host cell of embodiment 12, wherein the isolated BH comprises equivalent or increased activity relative to the activity of same BH heterologously expressed and isolated from an E. coli host cell.

24. A lysed host cell fermentation broth obtained from the host cell of embodiment 12.

25. A method for producing a heterologous cyanuric acid hydrolase (CAH) and/or a heterologous biuret hydrolase (BH) in a host cell, the method comprising: (a) obtaining a suitable host cell and introducing into the host cell a polynucleotide expression cassette encoding a heterologous CAH and/or introducing into the host cell a polynucleotide expression cassette encoding a heterologous BH, (b) fermenting the host cell under suitable conditions for the expression and production of the heterologous CAH and/or the heterologous BH, and (c) isolating the heterologous CAH and/or heterologous BH from the host cell or the host cell fermentation broth.

26. The method of embodiment 25, wherein the expression cassette encoding the CAH is derived from a parental cell selected from the group consisting of M. thermoacetica, B. diazoefficiens, Bradyrhizobium sp. (WSM1253), Pseudolabrys sp. (Root1462) and A. citrulli (122227) and/or wherein the heterologous polynucleotide encoding the BH is derived from a Herbaspirillum sp. (BH-1) parental cell.

27. The method of embodiment 25, wherein the CAH encoded by the heterologous polynucleotide comprises 45% or greater amino acid sequence identity to any one of SEQ ID NO: 2, SEQ ID NO: 6, SEQ ID NO: 8, SEQ ID NO: 10 and SEQ ID NO:12.

28. The method of embodiment 25, wherein the CAH encoded by the heterologous polynucleotide comprises SEQ ID NO: 23.

29. The method of embodiment 25, wherein the CAH encoded by the heterologous polynucleotide is an EC 3.5.2.15 enzyme.

30. The method of embodiment 25, wherein the BH encoded by the heterologous polynucleotide is an EC 3.5.1.84 enzyme.

31. The method of any one of embodiments 25-30, wherein the host cell is selected from a prokaryotic host cell and a eukaryotic microbial host cell.

32. The method of any one of embodiments 25-31, wherein the prokaryotic host cell excludes Gram-negative bacterial cells.

33. The method of any one of embodiments 25-32, wherein the host cell is selected from the group consisting of a Gram-positive bacterial cell, a filamentous fungal cell and a yeast cell.

34. The method of any one of embodiments 25-33, wherein the heterologous CAH expressed and produced is isolated from the host cell or the host cell fermentation broth, and/or wherein the heterologous BH expressed and produced is isolated from the host cell or the host cell fermentation broth.

35. An isolated CAH and/or an isolated BH produced by the method of embodiment 34.

36. An isolated CAH and/or isolated BH produced method of embodiment 34, wherein the isolated CAH comprises no detectable lipopolysaccharides (LPS) and/or the isolated BH comprises no detectable LPS.

37. An isolated CAH produced by the method embodiment 34, wherein the isolated CAH comprises equivalent or increased activity relative to the activity of same CAH endogenously produced and isolated from the parental cell from which the CAH gene was derived.

38. An isolated CAH produced by the method of embodiment 34, wherein the isolated CAH comprises equivalent or increased activity relative to the activity of same CAH heterologously expressed and isolated from an E. coli host cell.

39. An isolated CAH produced by the method of embodiment 34, wherein the isolated CAH does not comprises a poly-histidine tag.

40. An isolated BH produced by the method embodiment 34, wherein the isolated BH comprises equivalent or increased activity relative to the activity of same BH endogenously produced and isolated from the parental cell from which the BH gene was derived.

41. An isolated BH produced by the method of embodiment 34, wherein the isolated BH comprises equivalent or increased activity relative to the activity of same BH heterologously expressed and isolated from an E. coli host cell.

42. A method for producing a heterologous cyanuric acid hydrolase (CAH) and/or a heterologous biuret hydrolase (BH) in a host cell, the method comprising: (a) obtaining a suitable host cell and introducing into the host cell a polynucleotide expression cassette encoding a heterologous CAH and/or introducing into the host cell a polynucleotide expression cassette encoding a heterologous BH, (b) fermenting the host cell under suitable conditions for the expression and production of the heterologous CAH and/or the heterologous BH, (c) lysing the host cells after fermenting, and (d) at least partially purifying the CAH and/or BH from the lysed host cell fermentation broth.

43. The method of embodiment 42, wherein the partially purified the CAH and/or BH are substantially free of intact or viable host cell.

44. The method of embodiment 42, wherein the polynucleotide expression cassette encoding the CAH is derived from a parental cell selected from the group consisting of M. thermoacetica, B. diazoefficiens, Bradyrhizobium sp. (WSM1253), Pseudolabrys sp. (Root1462) and A. citrulli (122227) and/or wherein the heterologous polynucleotide encoding the BH is derived from a Herbaspirillum sp. (BH-1) parental cell.

45. The method of embodiment 42, wherein the polynucleotide expression cassette encoding the CAH comprises 45% or greater amino acid sequence identity to any one of SEQ ID NO: 2, SEQ ID NO: 6, SEQ ID NO: 8, SEQ ID NO: 10 and SEQ ID NO:12.

46. The method of embodiment 42, wherein the polynucleotide encoding the CAH comprises a nucleic acid sequence therein encoding SEQ ID NO: 23.

47. The method of embodiment 42, wherein the CAH encoded by the heterologous polynucleotide is an EC 3.5.2.15 enzyme.

48. The method of embodiment 42, wherein the BH encoded by the heterologous polynucleotide is an EC 3.5.1.84 enzyme.

49. The method of any one of embodiments 42-48, wherein the host cell is selected from a prokaryotic host cell and a eukaryotic microbial host cell.

50. The method of any one of embodiments 42-49, wherein the prokaryotic host cell excludes Gram-negative bacterial cells.

51. The method of any one of embodiments 42-49, wherein the host cell is selected from the group consisting of a Gram-positive bacterial cell, a filamentous fungal cell and a yeast cell.

52. A partially purified CAH and/or BH composition produced by the method of embodiment 42.

53. A partially purified CAH and/or BH composition produced by the method of embodiment 42, wherein the composition comprises no detectable LPS.

54. A partially purified CAH composition produced by the method of embodiment 42, wherein the composition comprises equivalent or increased activity relative to the activity of same CAH heterologously expressed and partially purified from an E. coli host cell.

55. A partially purified BH composition produced by the method of embodiment 42, wherein the composition comprises equivalent or increased activity relative to the activity of same BH heterologously expressed and partially purified from an E. coli host cell.

56. A partially purified CAH composition produced by the method of embodiment 42, wherein the composition comprises equivalent or increased activity relative to the activity of same CAH endogenously expressed and partially purified from the parental cell from which the CAH gene was derived.

57. A partially purified BH composition produced by the method of embodiment 42, wherein the composition comprises equivalent or increased activity relative to the activity of same BH endogenously expressed and partially purified from the parental cell from which the BH gene was derived.

### EXAMPLES

Certain aspects of the present disclosure may be further understood in light of the following examples, which should not be construed as limiting. Modifications to materials and methods will be apparent to those skilled in the art.

### Example 1

### Heterologous Production of Cyanuric Acid Hydrolases in Bacillus sp. Host Cells

Exemplary parental cells comprising an endogenous gene encoding a CAH, or comprising an endogenous gene encoding a BH, are generally described and set forth above in Section II. In the instant example, Applicant evaluated the heterologous expression/production of six (6) different genes encoding CAH enzyme homologues (e.g., see Table 1, Section II). More particularly, in the present example Bacillus subtilis was selected as an exemplary Bacillus sp. host cell for the heterologous expression of the six (6) different CAH genes. However, as described herein, the B. subtilis host cell is not meant to limit the choice of a host cell, as any other Bacillus sp. cell (or any other microbial host cell of the disclosure) may be adapted and used herein for the heterologous expression of a gene encoding a CAH.

Thus, as described herein, all six (6) of the heterologous CAH genes were integrated into the aprE locus on the B. subtilis genome (with no antibiotic resistance marker), wherein the genes (SEQ ID NO: 1, SEQ ID NO: 3, SEQ ID NO: 5, SEQ ID NO: 7, SEQ ID NO: 9 and SEQ ID NO: 11) encoding a CAH enzyme homologue, were codon-optimized for expression in a B. subtilis host and constructed using standard molecular biology techniques known to one skilled in the art.

For example, PCR products comprising the CAH genes (SEQ ID NO: 1, SEQ ID NO: 3, SEQ ID NO: 5, SEQ ID NO: 7, SEQ ID NO: 9 and SEQ ID NO: 11) were transformed into the B. subtilis host, as generally described in PCT Publication No. WO2018/187524 (incorporated herein by referenced in its entirety). One (1) μg of the PCR product was mixed with 200 μl of competent Bacillus cells comprising and expressing a comK gene (0.3% xylose-induced) and comprising deletion of alrA. The transformant was spread onto LB plates after incubation at 37° C., 250 rpm for one (1) hour. Single colonies were selected after incubation of the transformant at 37° C. overnight. The genomic region containing the gene of interest (i.e., SEQ ID NO: 1, SEQ ID NO: 3, SEQ ID NO: 5, SEQ ID NO: 7, SEQ ID NO: 9 or SEQ ID NO: 11) was confirmed by Sanger sequencing. Transformants expressing the heterologous CAH in B. subtilis were grown in a stirred aerobic bioreactor on LB medium. After growth at 30° C., 220 rpm for twenty-four (24) hours, fifty (50) μl of cells were harvested and lysed by 0.1 mg/ml of lysozyme at 37° C. for one (1) hour. Samples were mixed with 2×SDS sample buffer and boiled for ten (10) minutes before being analyzed by SD S-PAGE (data not shown).

The fermentation samples were analyzed for expression levels of CAH homologues by using a CAH activity assays as previously described (Seffernick et al., 2012) and determination of CYA levels in swimming pool water by UV absorbance, HPLC and melamine cyanurate precipitation were performed as previously described (Downes et al., 1984). Thus, the CAH genes screened for heterologous expression in a B. subtilis host included five (5) CAH homologues from distinct (parental) species (i.e., Moorella thermoacetica, Bradyrhizobium diazoefficiens, Pseudolabrys sp. (Root1462), Bradyrhizobium sp. (WSM1253), Acidovorax citrulli (122227), and one chlorite resistant engineered variant; Moorella thermoacetica C46A, as described in PCT Publication No. WO2016/141026A1.

### Example 2

### Isolation and Purification of Cyanuric Acid Hydrolases Heterologously Expressed from Bacillus sp. Host Cells

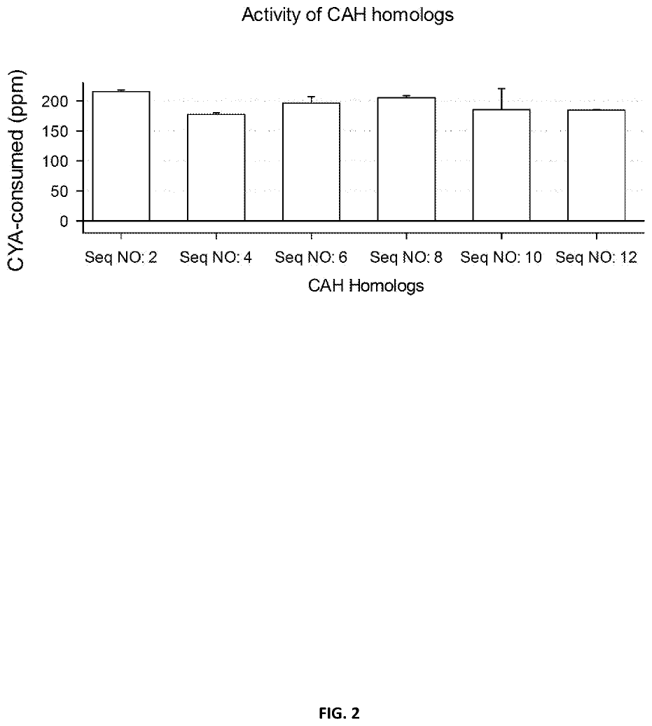

In the instant example, the B. subtilis host cells described above (e.g., comprising introduced polynucleotide expression cassettes encoding a heterologous CAH homologue (i.e., SEQ ID NO: 2. SEQ ID NO: 4, SEQ ID NO: 6, SEQ ID NO: 8, SEQ ID NO: 10 or SEQ ID NO: 12), were grown in a rich medium under glucose fed batch conditions. After the specific protein production phase of culture was reached, the fermentation was stopped. Standard large scale purification steps were implemented to isolate and purify the CAH homologues, which included filtration, the use of a polycationic polymer, and ultrafiltration (UF). This process is flexible and may proceed by any number of standard (scalable) purification procedures to yield a highly purified CAH homologue. The activity of each of the above purified CAH homologs are shown in FIG. 2. The measured CAH activity in the presence of 200 ppm cyanuric acid (CYA) of all the CAH homologues were diluted to a final concentration of approximately 0.3 mg/L and incubated in a solution of 200 ppm CYA at room temperature for sixteen (16) hours. Subsequently, the remaining unhydrolyzed CYA was measured and the consumed CYA was calculated by subtracting the remaining CYA from the starting 200 ppm CYA.

### Example 3

### Heterologous Production of Cyanuric Acid Hydrolases in Yeast Host Cells

Exemplary parental cells comprising an endogenous gene encoding a CAH, or comprising an endogenous gene encoding a BH, are generally described and set forth above in Section II. In the instant example, Applicant evaluated the heterologous expression of genes (SEQ ID NO: 5 and SEQ ID NO: 9) encoding CAH homologue of SEQ ID NO: 6 and SEQ ID NO: 10, respectively. More particularly, the present example describes the construction and optimization Saccharomyces cerevisiae host cells (an exemplary yeast cell) for the heterologous expression of two CAH (enzyme) homologues. However, as described herein, the S. cerevisiae cell is not meant to limit the choice of a host cell, as any other yeast cell (or any other microbial host cell of the disclosure) may be adapted and used herein for the heterologous expression of a gene encoding a CAH.

A. Galactose-Inducible Expression of CAH Homologs

The nucleic acid sequences of SEQ ID NO: 5 and SEQ ID NO: 9, encoding CAH homologues of SEQ ID NO: 6 and SEQ ID NO: 10, respectively, were obtained from a commercial gene synthesis company (Genewiz, South Plainfield, N.J.) each containing 5′ and 3′ end sequences to facilitate cloning into the commercially available yeast-E. coli shuttle vector pYES2 (Invitrogen catalog #V82520). The pYES2 vector contains the GAL1 promoter and the CYC1 terminator on either side of a multiple cloning site (MCS). After digestion of pYES2 with one or more restriction enzymes cutting in the MCS, the cut vector and commercially obtained CAH linear DNA (SEQ ID NO: 5 or SEQ ID NO: 9) were combined and transformed into a uracil auxotrophic S. cerevisiae host (PNY1500, also called BP857; described in U.S. Pat. No. 8,871,4880, incorporated herein by reference in its entirety) using standard lithium acetate transformation methods. Transformants are obtained via selection on synthetic complete medium lacking uracil (Catalog No. C3080, Teknova Inc., Hollister, Calif.).

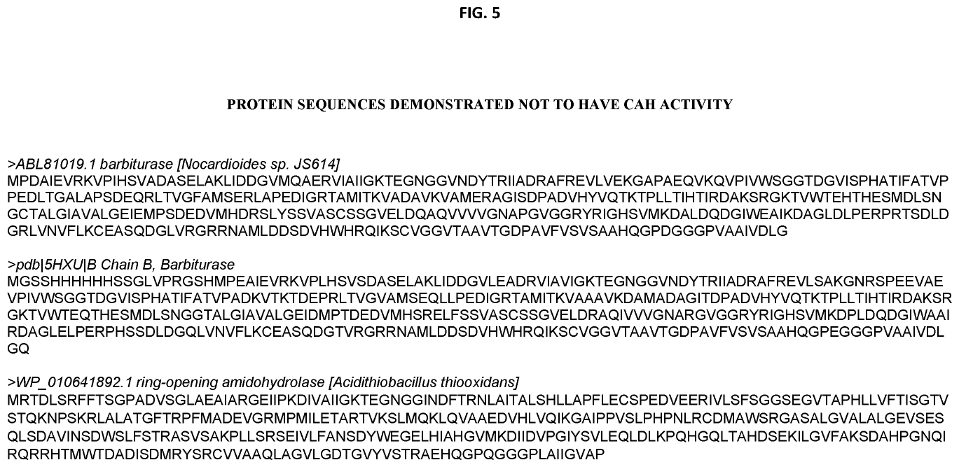

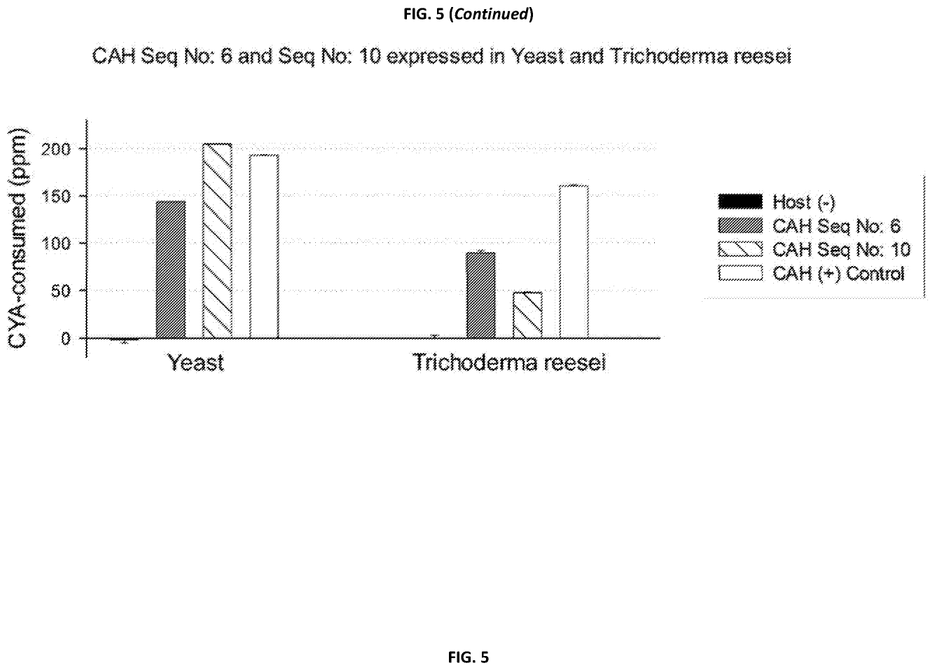

Proper assembly of the expression plasmids (pYES2::cah_SED ID NO: 5 and pYES2::cah_SEQ ID NO: 9) was confirmed using PCR primers CAH001 (SEQ ID NO: 15) and CAH002 (SEQ ID NO: 16) for CAH_Bj, and CAH001 (SEQ ID NO: 15) and CAH003 (SEQ ID NO: 17) for CAH_PR. Three clones each were grown in synthetic complete liquid medium without uracil (Teknova Catalog No. C8140) and then transferred to the same medium containing raffinose (2%) instead of glucose. After twenty (24) hours, cells were sub-cultured into the same medium containing galactose (2%). After twenty (24) hours, cells were recovered by centrifugation. Control cells/strains comprising the host cell/strain PNY1500 transformed with pYES2 were grown and collected in the same manner. Cell pellets were stored at ⁻80° C. Frozen yeast cells were thawed and resuspended in 0.1 M KPO₄ (pH 7.5) plus 100 mM sodium chloride. Yeast cells were broken by 2 passes in a French pressure cell. The extracts were held at 4° C. on ice and assayed for CAH activity (Table 2 and FIG. 5) within a few hours.

TABLE 2

| CAH Expressed in Yeast | Activity (mg/h/ml) |
|---|---|
| Empty Vector (−) control | −0.011 ± 0.022 |
| CAH SEQ ID NO: 6 | 0.898 ± 0.001 |
| CAH SEQ ID NO: 10 | 1.282 ± 0.001 |
| CAH (+) control (Bacillus host) | 1.206 ± 0.002 |

B. Constitutive Expression of CAH Homologues

The CAH genes obtained from commercial synthesis (described above) were amplified by PCR using primers that add 5′ and 3′ sequences to the ends that are compatible with another vector, pJT257 (see, PCT Publication No. WO2014/151645, vector sequence SEQ ID NO: 105). Use of this vector will provide for the expression of CAH genes using the strong, constitutive FBA1 promoter. The terminator also corresponds to that of the FBA1 gene. After digestion of pJT257 with SpeI and NotI, the 6.4 kb vector band is isolated and purified after agarose gel electrophoresis, recovered using a Zymoclean Gel DNA Recovery Kit (Catalog No. D4001, Zymo Research, Irvine, Calif.). The CAH genes were PCR amplified with primers CAH004 (SEQ ID NO: 18) and CAH005 (SEQ ID NO: 19) or CAH006 (SEQ ID NO: 20) and CAH007 (SEQ ID NO: 21), for CAH_Bj (SEQ ID NO: 5) and CAH_PR (SEQ ID NO: 9), respectively. Cut vector and PCR amplified CAH gene were combined and transformed into strain PNY1500, as described above. Proper assembly of the expression plasmids (pJT257::cah_Bj and pJT257::cah_PR) was confirmed using PCR primers CAH008 (SEQ ID No: 22) and CAH002 (SEQ ID NO: 16) for CAH_Bj and CAH008 (SEQ ID NO: 22) and CAH003 (SEQ ID NO: 17) for CAH_PR. Transformants were evaluated for production CAH in shake flasks as described above, except that cells need only be grown in synthetic complete medium lacking uracil, and do not need to be transferred to raffinose or galactose for activation of the promoter.

### Example 4

### Heterologous Production of Cyanuric Acid Hydrolases in Filamentous Fungal Cells

Exemplary parental cells comprising an endogenous gene encoding a CAH, or comprising an endogenous gene encoding a BH, are generally described and set forth above in Section II. In the instant example, Applicant evaluated the heterologous expression of a genes (SEQ ID NO: 5 and SEQ ID NO: 9) encoding CAH homologues of SEQ ID NO: 6 and SEQ ID NO: 10, respectively. More particularly, the present example describes the construction and optimization of Trichoderma reesei host cells (an exemplary filamentous fungal host cell) for the heterologous expression of a CAH (enzyme) homologue. However, as described herein, the T. reesei cell is not meant to limit the choice of a host cell, as any other filamentous fungal cell (or any other microbial host cell of the disclosure) may be adapted and used herein for the heterologous expression of a gene encoding a CAH.

Thus, as described herein, a series of expression cassettes were constructed to express a codon-optimized CAH proteins of SEQ ID NO: 6 or SEQ ID NO: 10 in T. reesei host cells, presented below in Table 3 and FIG. 5.

TABLE 3

| CAH expressed in T. reesei | Activity (mg/h/ml) |
|---|---|
| Host (−) | −0.000 ± 0.300 |
| CAH SEQ NO: 6 | 8.989 ± 0.193 |
| CAH SEQ NO: 10 | 4.752 ± 0.088 |
| CAH (+) Control (Bacillus host) | 16.060 ± 0.064 |

### Example 5

### A Novel Amino Acid Consensus Sequence to Identify Proteins Comprising Cyanuric Acid Hydrolase Activity

Cyanuric acid hydrolase (CAH) enzymes are members of a protein family that includes barbiturase, and other proteins that have been shown not to react with cyanuric acid or barbituric acid. For example, all members of the protein family, in a BLAST algorithm pairwise sequence alignment with any other member of the family, show amino acid sequence identity of at least 30%, and an e-value of e⁻¹⁰ (or lower). In light of this, the use of a BLAST search algorithm, or other standard protein sequence analysis methods, do not firmly identify a protein as a cyanuric acid hydrolase (i.e., a protein comprising CAH activity).

In the instant example, Applicant has analyzed the primary (1°) amino acid sequence of known proteins comprising CAH activity and identified a novel amino acid sequence motif (e.g., SEQ ID NO: 23) conserved among all proteins known to comprise CAH activity. As shown in FIG. 3, the identified consensus sequence of SEQ ID NO: 23 is a collection of sixteen (16) consecutive amino acid residues in the primary (1°) sequence, wherein the amino acid “Xaa” in the first (1) position of SEQ ID NO: 23 is a “Tyr” or “Phe”, the amino acid “Xaa” in the second (2) position of SEQ ID NO: 23 may be any amino acid, the amino acid “Xaa” in the sixth (6) position of SEQ ID NO: 23 may be any amino acid, the amino acid “Xaa” in the eight (8) position of SEQ ID NO: 23 is a “His” or “Asn”, the amino acid “Xaa” in the twelfth (12) position of SEQ ID NO: 23 may be any amino acid, and the amino acid “Xaa” in the six-tenth (16) position of SEQ ID NO: 23 is a “Pro” or “Ser”. More particularly, the serine (S) residue (FIG. 3, position 3) of the SEQ ID NO: 23 consensus sequence is one of the conserved serine residues in the active site of proteins comprising CAH activity, and other amino acid residues of the SEQ ID NO: 23 consensus sequence are in the region of the bound metal residue that has been identified in the X-ray structures of all cyanuric acid hydrolase to date (Bera et al., 2017; Shi et al., 2019).

Applicant further tested and validated the consensus sequence of SEQ ID NO: 23 by sequence comparison/analysis to proteins known to have CAH activity (FIG. 4) versus proteins known not to have CAH activity (FIG. 5). For example, the names associated with a specified protein in FIG. 4 and FIG. 5 come from genome annotations, which annotations are often incorrect, reflecting the difficulty for one skilled in the art in discerning cyanuric acid hydrolases versus barbiturase and/or general ring opening amidohydrolases. More particularly, the proteins comprising the specific amino acid sequences presented in FIG. 4 have an exact match to the consensus sequence (SEQ ID NO: 23) at their C-terminus and such proteins have been experimentally verified by Applicant, or in the scientific literature, to comprise CAH activity. In contrast, the proteins comprising the specific amino acid sequences presented in FIG. 5 have been experimentally verified by Applicant, or in the scientific literature, as not having (not comprising) CAH activity, wherein these proteins not having CAH activity do not have an exact match to the consensus sequence (SEQ ID NO: 23) at their C-terminus. The novel consensus sequence of SEQ ID NO: 23 is therefore particularly useful in identifying proteins (enzymes) comprising CAH activity, which proteins comprising CAH activity are particularly suitable for use in the CYA remediation methods and compositions of the instant disclosure.

### REFERENCES

PCT International Publication No. WO2007/107981
PCT International Publication No. WO2011/075677
PCT International Publication No. WO2016/130523
PCT International Publication No. WO1996/00787
PCT International Publication No. WO2016/141026
PCT International Publication No. WO2016/141026
PCT International Publication No. WO2017/019867
PCT International Publication No. WO2017/021966
PCT International Publication No. WO2018/093752
PCT International Publication No. WO2018/187524
U.S. Pat. No. 8,303,964
U.S. Pat. No. 8,871,480
U.S. Patent Publication No. US2013/0186837
U.S. Patent Publication No. US2018/0257963
Bera et al., “Structure of the Cyanuric Acid Hydrolase TrzD Reveals Product Exit Channel”, Sci. Rep., (7), Mar. 27; 2017.
Botstein and Shortie, Science 229: 4719, 1985.
Burke et al., CSHL Press, 103-105, 2000.
Dohmen et al., Yeast, 7: 691-692, 1991.
Downes et al., “Determiniation of cyanuric acid levels in swimming pool waters by UV absorbance, HPLC and melamine cyanurate precipitation”, Water Research 18:277-280, 1984.
Esquirol et al., “A novel decarboxylating amidohydrolase involved in avoiding metabolic dead ends during cyanuric acid catabolism in Pseudomonas sp. strain ADP”, PLoS One, 13(11), 2018a.
Esquirol et al., “Structural and biochemical characterization of the biuret hydrolase (BiuH) from the cyanuric acid catabolism pathway of Rhizobium leguminasorum bv. viciae 3841”, PLoS One, 13(2), 2018b.
Higuchi et al., Nucleic Acids Research 16: 7351, 1988.
Ho et al., Gene 77: 61, 1989.
Horton et al., Gene 77: 61, 1989.
Liu and Zuber, “A Molecular Switch Controlling Competence and Motility: Competence Regulatory Factors ComS, MecA, and ComK Control Dependent Gene Expression in Bacillus subtilis”, Journal of Bacteriology 180 (16): 4243-4251, 1998.
Lo et al., Proceedings of the National Academy of Sciences USA 81:2285, 1985.
Malardier et al., Gene 78: 147-156, 1989.
Mamat et al., “Detoxifying Escherichia coli for Endotoxin-Free Production of Recombinant Proteins”, Microbial Cell Factories, 14(57): 1-15, 2015.
Nevoigt, “Progress in Metabolic Engineering of Saccharomyces cerevisiae”, Microbiology and Molecular Biology Reviews, 72(3): 379-412, 2008.
Ostergaard et al., “Metabolic Engineering of Saccharomyces cerevisiae”, Microbiology and Molecular Biology Reviews, 64(1): 34-50, 2000.
Petes et al., “Recombination in yeast and the recombinant DNA technology”, Genome, 31(2): 536-540, 1989.
Protein Purification, J. C. Janson and Lars Ryden, editors, VCH Publishers, New York, 1989.
Sambrook, Fritsch, and Maniatus, “Molecular Cloning, A Laboratory Manual, 2^{nd} edition”, Cold Spring Harbor, N.Y., 1989.
Sarkar and Sommer, BioTechniques 8: 404, 1990.
Seffernik et al., “Defining Sequence Space and Reaction Products within the Cyanuric Acid Hydrolase (AtzD)/Barbiturase Protein Family”, J. Bacteriology, 194(17): 4579-4588, 2012.
Shi et al., “Crystal structures of Moorella thermoacetica cyanuric acid hydrolase reveal conformational flexibility and asymmetry important for catalysis”, PLoS ONE 14(6), 2019.
Shimada, Meth. Mol. Biol. 57: 157, 1996.
Walsh, “Biopharmaceutical Benchmarks 2010”, Nat. Biotechnol., 28: 917-924, 2010.
Yeom et al., “Bacterial Cyanuric Acid Hydrolase for Water Treatment”, Appl. Environ. Microbiol., October; 81(19): 6660-6668, 2015.

1. A host cell expressing a heterologous polynucleotide encoding a cyanuric acid hydrolase (CAH) and/or expressing a heterologous polynucleotide encoding a heterologous biuret hydrolase (BH).

2. The host cell of claim 1, wherein the heterologous polynucleotide encoding the CAH is derived from a parental cell selected from the group consisting of M. thermoacetica, B. diazoefficiens, Bradyrhizobium sp. (WSM1253), Pseudolabrys sp. (Root1462) and A. citrulli (122227) and/or wherein the heterologous polynucleotide encoding the BH is derived from a Herbaspirillum sp. (BH-1) parental cell.

3. The host cell of claim 1, wherein the CAH encoded by the heterologous polynucleotide comprises 45% or greater amino acid sequence identity to any one of SEQ ID NO: 2, SEQ ID NO: 6, SEQ ID NO: 8, SEQ ID NO: 10 and SEQ ID NO:12.

4. The host cell of claim 1, wherein the CAH encoded by the heterologous polynucleotide comprises SEQ ID NO: 23.

5. The host cell of claim 1, selected from the group consisting of a Gram-positive bacterial cell, a filamentous fungal cell and a yeast cell.

6-11. (canceled)

12. A lysed host cell fermentation broth obtained from the host cell claim 1.

13. A method for producing a heterologous cyanuric acid hydrolase (CAH) and/or a heterologous biuret hydrolase (BH) in a host cell, the method comprising:
(a) obtaining a suitable host cell and introducing into the host cell a polynucleotide expression cassette encoding a heterologous CAH and/or introducing into the host cell a polynucleotide expression cassette encoding a heterologous BH,
(b) fermenting the host cell under suitable conditions for the expression and production of the heterologous CAH and/or the heterologous BH, and
(c) isolating the heterologous CAH and/or heterologous BH from the host cell or the host cell fermentation broth.

14. The method of claim 13, wherein the expression cassette encoding the CAH is derived from a parental cell selected from the group consisting of M. thermoacetica, B. diazoefficiens, Bradyrhizobium sp. (WSM1253), Pseudolabrys sp. (Root1462) and A. citrulli (122227) and/or wherein the heterologous polynucleotide encoding the BH is derived from a Herbaspirillum sp. (BH-1) parental cell.

15. The method of claim 13, wherein the CAH encoded by the heterologous polynucleotide comprises 45% or greater amino acid sequence identity to any one of SEQ ID NO: 2, SEQ ID NO: 6, SEQ ID NO: 8, SEQ ID NO: 10 and SEQ ID NO:12.

16. The method of claim 13, wherein the CAH encoded by the heterologous polynucleotide comprises SEQ ID NO: 23.

17. The method of claim 13, wherein the host cell is selected from the group consisting of a Gram-positive bacterial cell, a filamentous fungal cell and a yeast cell.

18. (canceled)

19. (canceled)

20. The method of claim 13, wherein the isolated CAH comprises equivalent or increased activity relative to the activity of same CAH endogenously produced and isolated from the parental cell from which the CAH gene was derived.

21. The method of claim 13, wherein the isolated CAH comprises equivalent or increased activity relative to the activity of same CAH heterologously expressed and isolated from an E. coli host cell.

22. The method claim 13, wherein the isolated BH comprises equivalent or increased activity relative to the activity of same BH endogenously produced and isolated from the parental cell from which the BH gene was derived.

23. The method claim 13, wherein the isolated BH comprises equivalent or increased activity relative to the activity of same BH heterologously expressed and isolated from an E. coli host cell.

24. A method for producing a heterologous cyanuric acid hydrolase (CAH) and/or a heterologous biuret hydrolase (BH) in a host cell, the method comprising:
(a) obtaining a suitable host cell and introducing into the host cell a polynucleotide expression cassette encoding a heterologous CAH and/or introducing into the host cell a polynucleotide expression cassette encoding a heterologous BH,
(b) fermenting the host cell under suitable conditions for the expression and production of the heterologous CAH and/or the heterologous BH,
(c) lysing the host cells after fermenting, and
(d) at least partially purifying the CAH and/or BH from the lysed host cell fermentation broth.

25. The method of claim 24, wherein the partially purified the CAH and/or BH are substantially free of intact or viable host cell.

26. The method of claim 24, wherein the polynucleotide expression cassette encoding the CAH is derived from a parental cell selected from the group consisting of M. thermoacetica, B. diazoefficiens, Bradyrhizobium sp. (WSM1253), Pseudolabrys sp. (Root1462) and A. citrulli (122227) and/or wherein the heterologous polynucleotide encoding the BH is derived from a Herbaspirillum sp. (BH-1) parental cell.

27. The method of claim 24, wherein the polynucleotide expression cassette encoding the CAH comprises 45% or greater amino acid sequence identity to any one of SEQ ID NO: 2, SEQ ID NO: 6, SEQ ID NO: 8, SEQ ID NO: 10 and SEQ ID NO:12.

26. The method of claim 24, wherein the polynucleotide encoding the CAH comprises a nucleic acid sequence therein encoding SEQ ID NO: 23.

27. The method of claim 24, wherein the host cell is selected from the group consisting of a Gram-positive bacterial cell, a filamentous fungal cell and a yeast cell.

28. (canceled)

29. (canceled)

30. A partially purified CAH and/or BH composition produced by the method of claim 24, wherein the composition comprises equivalent or increased activity relative to the activity of same CAH heterologously expressed and partially purified from an E. coli host cell and/or wherein the composition comprises equivalent or increased activity relative to the activity of same BH heterologously expressed and partially purified from an E. coli host cell.

31. (canceled)

32. A partially purified CAH and/or BH composition produced by the method of claim 24, wherein the composition comprises equivalent or increased activity relative to the activity of same CAH endogenously expressed and partially purified from the parental cell from which the CAH gene was derived and/or wherein the composition comprises equivalent or increased activity relative to the activity of same BH endogenously expressed and partially purified from the parental cell from which the BH gene was derived.

33. (canceled)


SEQUENCE LISTING (23 sequences)

SEQ ID NO: 1 - DNA, 1104 bp, Moorella thermoacetica
atgcaaaaagttgaagtctttcgtatcccaacagcctcacctgatgatatcagcgggttggctactttgattgactcagggaagattaatccggctgaaattgtagctattctgggcaagacggaaggtaatggttgtgttaacgattttactcgcggttttgctacccaatctctagcaatgtatctagcagaaaaattgggaataagccgcgaagaagttgtaaaaaaagttgcgtttataatgtcaggcggtactgagggtgttatgacgccgcacattactgtctttgtccgaaaagatgtccaggaaccagctaagccaggaaagcgcttggcagtaggcgtagcttttacgcgggactttctgccggaagaattgggccggatggaacaggtaaacgaagtggctcgagctgtaaaagaggctatgaaagacgcccaaatagatgatccccgcgacgttcactttgttcagataaagtgcccgcttttgactgctgaaagaattgaggatgccaagcgacgcgggaaagatgttgtggtaaatgacacttataaatcaatggcttactcacgtggcgcctcagctctcggagtggccctcgcactgggcgagatatctgcggataagattagcaacgaagcaatttgtcatgactggaatctctactctagtgtggcttcgacgtcagcgggggttgaacttcttaatgacgaaattatcgtagtaggaaactctaccaatagtgccagtgatttggttataggccactctgttatgaaggatgccattgacgctgatgcagtacgtgctgcccttaaagatgccggtttaaaatttgattgttgcccgccagcagaagagcttgctaagatcgttaatgtactggccaaggctgaggccgcatcttctggtacagtaaggggtaggcgtaacacgatgcttgatgactctgatatcaatcatacccgctcggcgcgagcagtagtaaatgctgttattgccagcgtggtaggagatcctatggtctacgtttccggtggtgcagagcatcaagggcctgatggcggtggcccaattgctgttattgccagggtgtag

SEQ ID NO: 2 - protein, 367 aa, Moorella thermoacetica
MQKVEVFRIPTASPDDISGLATLIDSGKINPAEIVAILGKTEGNGCVNDFTRGFATQSLAMYLAEKLGISREEVVKKVAFIMSGGTEGVMTPHITVFVRKDVQEPAKPGKRLAVGVAFTRDFLPEELGRMEQVNEVARAVKEAMKDAQIDDPRDVHFVQIKCPLLTAERIEDAKRRGKDVVVNDTYKSMAYSRGASALGVALALGEISADKISNEAICHDWNLYSSVASTSAGVELLNDEIIVVGNSTNSASDLVIGHSVMKDAIDADAVRAALKDAGLKFDCCPPAEELAKIVNVLAKAEAASSGTVRGRRNTMLDDSDINHTRSARAVVNAVIASVVGDPMVYVSGGAEHQGPDGGGPIAVIARV

SEQ ID NO: 3 - DNA, 1104 bp, Moorella thermoacetica
atgcaaaaagtcgaagtttttagaatcccaacggctagccccgatgatatttcaggactagcgaccttaattgatagtggaaaaatcaatccggcagagattgttgcaattttgggaaaaactgaagggaatggtgctgtgaacgattttactagaggattcgcgacgcaatccttagctatgtatttagccgaaaagttaggtattagtcgtgaggaagttgtcaaaaaagtcgcattcattatgtctggtggaactgagggtgtaatgactccacatattacggtctttgtccggaaagacgtacaagagccggccaaacctggaaaacggttagcggtgggtgttgcgtttacccgtgatttcttgccggaagaactcggacgcatggaacaagtcaacgaagtcgccagggctgtaaaagaggccatgaaagatgcccagattgatgacccgcgagatgtacattttgtccaaatcaaatgtccgttactgacagcggagcgtattgaagatgcaaaacgtcgcggtaaagatgtagtagtaaacgacacctacaagtcaatggcgtactcccgtggggcatcagcgttaggtgtagcattagcgctcggtgagatttctgcggataagatttctaatgaagccatttgccatgattggaatctatatagctcggtcgcctccacaagtgccggagtagagttactaaacgatgaaatcattgtagtcggcaacagtactaattctgcgagtgatcttgtaattggacactctgtaatgaaagatgcaattgatgcggacgcagtcagggcagcactgaaagatgcgggactgaaatttgattgttgtccaccggcagaggaattagcgaaaattgttaatgtattagccaaagctgaggcagcgtcaagtggaacggtgcgtggacgccgtaacactatgttagacgacagcgacattaatcatacgcgcagcgctcgcgccgtagtcaatgccgtcattgcttccgtcgttggtgatccaatggtatatgtcagtggtggagcagagcaccagggacccgacggtgggggaccgattgcggtaattgcgcgtgtttag

SEQ ID NO: 4 - protein, 367 aa, Moorella thermoacetica
MQKVEVFRIPTASPDDISGLATLIDSGKINPAEIVAILGKTEGNGAVNDFTRGFATQSLAMYLAEKLGISREEVVKKVAFIMSGGTEGVMTPHITVFVRKDVQEPAKPGKRLAVGVAFTRDFLPEELGRMEQVNEVARAVKEAMKDAQIDDPRDVHFVQIKCPLLTAERIEDAKRRGKDVVVNDTYKSMAYSRGASALGVALALGEISADKISNEAICHDWNLYSSVASTSAGVELLNDEIIVVGNSTNSASDLVIGHSVMKDAIDADAVRAALKDAGLKFDCCPPAEELAKIVNVLAKAEAASSGTVRGRRNTMLDDSDINHTRSARAVVNAVIASVVGDPMVYVSGGAEHQGPDGGGPIAVIARV

SEQ ID NO: 5 - DNA, 1113 bp, Bradyrhizobium diazoefficiens
atgagaacaacatcagttggcgtgtttaagatcgttacaaaaggaccgggtgatgtttcaggccttatggcaatgattggctcaggcgcaattgatccgaaatcaattctggcagttctgggcaaaacggaaggcaatggcggagttaatgattttacaagagaatatgcagttgcagcactttgcacagcactggcaccgcaactgggcctgtcaccggaagaagttgaacaaagaattgcatttgttatgagcggaggcacagaaggcgttctgtcaccgcatattacagtctttacaagaagagaagtcgaacgcagaccggcaggcctgtcaggcaaaagactgtcaattggcatggcacatacaagagattttctgccggaagaactgggcagagcagcacaaattgcagaaacagcagcagcagttaaagcagcaatggcagatgcaggcattgcagatccggcagatgttcattttgttcagattaaatgtccgctgctgacaagcgatagagttgaagcagcatcagcaagaggcaataaaacagcaacaacaagcgcatatggctcaatggcatattcaagaggcgcatcagcactgggcgttgcagttgcgctgggcgaaacaggctcagatatttcagatggcgacgttctgagaagatatgacctgttttcaaaagttgcgtcaacatcagcgggaattgaactgatgcataatgttgttattgtcctgggcaattcagcagcaagcgcatcagaatttgaaattggccatgcagttatgaacgatgcaattgatgcagcagcggttacatcagcgcttaaatgcgttggccttggcgttgcaccgcaagcagaagcaggcagagaactggttaatatctttgcaaaagcggaagcatcaccggatggctcagttagaggctttagacatacaatgctggaagatacagatattagctcaacaagacatgcaagagcagcagtcggaggcctgattgcaggactggcaggcacaggcgcagtttatgtttctggcggagcagaacatcaaggaccggctggcggaggaccggttgcagttattgcaagactgagcgattaa

SEQ ID NO: 6 - protein, 370 aa, Bradyrhizobium diazoefficiens
MRTTSVGVFKIVTKGPGDVSGLMAMIGSGAIDPKSILAVLGKTEGNGGVNDFTREYAVAALCTALAPQLGLSPEEVEQRIAFVMSGGTEGVLSPHITVFTRREVERRPAGLSGKRLSIGMAHTRDFLPEELGRAAQIAETAAAVKAAMADAGIADPADVHFVQIKCPLLTSDRVEAASARGNKTATTSAYGSMAYSRGASALGVAVALGETGSDISDGDVLRRYDLFSKVASTSAGIELMHNVVIVLGNSAASASEFEIGHAVMNDAIDAAAVTSALKCVGLGVAPQAEAGRELVNIFAKAEASPDGSVRGFRHTMLEDTDISSTRHARAAVGGLIAGLAGTGAVYVSGGAEHQGPAGGGPVAVIARLSD

SEQ ID NO: 7 - DNA, 1113 bp, Bradyrhizobium sp.
atgagaacaacaagcgttggcgtttttaaagttgcaacaaaaggaccgggtgatgtttcaggccttatggcaatgattggctcaggcgcaattgatccggcaagcattctggcaattctgggcaaaacggaaggcaatggcggagttaatgattttacaagagaatatgcagttgcagcactttgcacagcactggcaccgcaactgggcctgtcaccgcaagaagttgaacaaagaattgcatttgttatgagcggaggcacagaaggcgttctttcaccgcatattacagtctttacaagaagagatgttctgcaaagaccggcaggcatttcaggcaaaagactgtcaattggcatggcacatacaagagattttttaccggaagaactgggcagatcagcacaaatcacagaaacagcaaaagcagttaaagcagcaatggcagatgcaggcattgcagatccggcagatgttcattttgttcagattaaatgtccgctgctgacatcagaacgcgttgaagcagcaaatgcaagaggccataaaacagcaacaacatcagcatattcaagcatggcatatagcagaggcgcatcagcactgggcgttgcagttgcgctgggcgaaattgcgacagatctgagagatgatgatgtcctgagaagatatgacctgttttcaaatgttgcatcaacaagctcaggcatcgaactgacacataatgttgttattgttctcggcaatagcatgtcaagcacaagcgaatttgaaattggccatgcagttatgtcagatgcaattgatgcagcagcagttcttgcagcgctggaatcagttggcctgtgcgcagcaccgcaaacaacaacaggcagagaactggttaatatctttgcaaaagcggaagcatcaccggatggctcagttagaggctttagacatacaatgctggaagatacagatatttcatcaacacgccatgcaagagcagcagtcggaggcctgattgcaggcctggcaggcacaggcgcagtttatgtttctggcggagcggaacatcaaggaccggctggcggaggaccggttgcagttattgcaaaacttagcgattaa

SEQ ID NO: 8 - protein, 370 aa, Bradyrhizobium sp.
MRTTSVGVFKVATKGPGDVSGLMAMIGSGAIDPASILAILGKTEGNGGVNDFTREYAVAALCTALAPQLGLSPQEVEQRIAFVMSGGTEGVLSPHITVFTRRDVLQRPAGISGKRLSIGMAHTRDFLPEELGRSAQITETAKAVKAAMADAGIADPADVHFVQIKCPLLTSERVEAANARGHKTATTSAYSSMAYSRGASALGVAVALGEIATDLRDDDVLRRYDLFSNVASTSSGIELTHNVVIVLGNSMSSTSEFEIGHAVMSDAIDAAAVLAALESVGLCAAPQTTTGRELVNIFAKAEASPDGSVRGFRHTMLEDTDISSTRHARAAVGGLIAGLAGTGAVYVSGGAEHQGPAGGGPVAVIAKLSD

SEQ ID NO: 9 - DNA, 1092 bp, Pseudolabrys sp.
atgccgattgcaaaagtccatagaatttcagcatcaagcccgaatgatgtttcaggcctggaagcagcaattgcatcaggcagaattgatccgaaaggcgttattgcagttctgggcaaaacagaaggcaacggcctggttaatgatttttcaagaggcctggcaacaacagcactgacactgctgtttgaaagacatctgccgcaagcagaagcagcacaaatttgcctggttatgtcaggcggaacggaaggcggaatggcaccgcattggattgtttttgaaagaggcaaaggcgaaggcggacgctcaccggcactggcaattggcagagcacatacactgcaactgccgtatgaacaactgggcagactgggcgaagttgatcaagttgcagcaggcgttagagcagcaatggaagatgcaggcattgcagatccggctgatgttcattttgttcagattaaatgtccgctgctgacagcacaaagaattgcagaagcggaagcaagaggcgcagcagttgcaacaagagatacactgaaatcaatgtcactgtcaagagcagcatcagcactgggagcagcagtcgcacttggcgaaattgatagagcagcgattacagaagcgcaaattggctcagattggtcactgtggtcaggcagagcatcaacatcagctggcattgaactggtcaatcatgaaattgttgttctcggcatgagcaaagattggagcggaccgcttgcaattgatcatgcagttatgagagatggcattgatattgaaccggttagagcggcactgcaaagacttggccttggcgcagcgggacaactggatccggcacaacgcaataaaatggttgcactgctggcaaaagctgaagcatcacatgatggcagactgagaggccatagacatacaatgctggatgattcagatattgcgtcaacaagacatgcaagaggctttgtttgcggagcactggcaggcctggttggccatgcagaaatttatgttagcggaggcgcagaacatcaaggaccggatggcggaggaccggttgcagttattgttgatagaggctaa

SEQ ID NO: 10 - protein, 363 aa, Pseudolabrys sp.
MPIAKVHRISASSPNDVSGLEAAIASGRIDPKGVIAVLGKTEGNGLVNDFSRGLATTALTLLFERHLPQAEAAQICLVMSGGTEGGMAPHWIVFERGKGEGGRSPALAIGRAHTLQLPYEQLGRLGEVDQVAAGVRAAMEDAGIADPADVHFVQIKCPLLTAQRIAEAEARGAAVATRDTLKSMSLSRAASALGAAVALGEIDRAAITEAQIGSDWSLWSGRASTSAGIELVNHEIVVLGMSKDWSGPLAIDHAVMRDGIDIEPVRAALQRLGLGAAGQLDPAQRNKMVALLAKAEASHDGRLRGHRHTMLDDSDIASTRHARGFVCGALAGLVGHAEIYVSGGAEHQGPDGGGPVAVIVDRG

SEQ ID NO: 11 - DNA, 1113 bp, Acidovorax citrulli
atgcaagcgcaagtttttagagttccgatgtcaaatccggcagatgtttcaggcgttgcaaaactgattgatgaaggcgttattagagcagaagaagttgtttgcgttctgggcaaaacagaaggcaacggctgcgttaatgattttacaagaggctatacaacgctggcgtttaaagtctacttttcagaaaaactgggcgtgagcagacaagaagttggcgaaagaattgcgtttattatgtcaggcggaacggaaggcgtcatggcaccgcattgcacaatttttacagttcagaaaacggataacaaacagaaaacagcagcggaaggcaaaagactggcagttcagcagatttttacacgcgaatttttgccggaagaaattggcagaatgccgcaagttacagaaacagcggatgcagttagacgcgcaatgagagaagcaggcattgcagatgcatcagatgttcattttgtccaagttaaatgtccgctgcttacagcgggaagaatgcatgatgcagttgaaagaggccatacagttgcaacggaagatacatatgaatcaatgggctattcaagaggcgcatcagcactgggcattgcactggcactgggcgaagttgaaaaagcaaatctgtcagatgaagtcatcacagcagattattcactgtattcatcagttgcatcaacaagcgctggcattgaattgatgaacaacgaaattattgtcatgggcaatagcagagcatggggaggcgatctggttattggccatgcagaaatgaaagatgcaattgatggcgcagcagtcagacaagcactgagagatgttggctgctgcgaaaatgatctgccgacagttgatgaactgggcagagttgttaatgtttttgcgaaagcggaagcatcaccggatggcgaagtcagaaatagacgccatacaatgctggatgatagcgatattaacagcacaagacatgcaagagcagttgtcaatgcagttattgcatcaattgttggcgatccgatggtttatgttagcggaggctcagaacatcaaggaccggcaggcggaggaccggttgcagtcattgcaagaacagcataa

SEQ ID NO: 12 - protein, 370 aa, Acidovorax citrulli
MQAQVFRVPMSNPADVSGVAKLIDEGVIRAEEVVCVLGKTEGNGCVNDFTRGYTTLAFKVYFSEKLGVSRQEVGERIAFIMSGGTEGVMAPHCTIFTVQKTDNKQKTAAEGKRLAVQQIFTREFLPEEIGRMPQVTETADAVRRAMREAGIADASDVHFVQVKCPLLTAGRMHDAVERGHTVATEDTYESMGYSRGASALGIALALGEVEKANLSDEVITADYSLYSSVASTSAGIELMNNEIIVMGNSRAWGGDLVIGHAEMKDAIDGAAVRQALRDVGCCENDLPTVDELGRVVNVFAKAEASPDGEVRNRRHTMLDDSDINSTRHARAVVNAVIASIVGDPMVYVSGGSEHQGPAGGGPVAVIARTA

SEQ ID NO: 13 - DNA, 696 bp, Herbaspirillum sp.
atgcctgaactcttcatcaaagccgaaccctatgcatggccctatgacggcgcactgaccccggccaataccgcactcatcgtcatcgacatgcagaccgacttctgcggcatcggcggctacgtcgacaagatgggctacgacctgtcattgacgcgcgctcccatcgagccgatcaagcgcgtgctggccgccatgcgcgccgggggctataccatcatccatacccgcgaaggccaccgtcccgatctctccgacctgcccgccaacaagcgctggcgttcgcgccagatcggtaccaatggcgtgggcatcggtgacgctggtccgtgtggccgcatcctggtgcggggtgaaccgggctgggaaatcattcccgaactggcccccatcgccggcgagatcatcatcgacaaacccggcaagggttccttctgcgccaccgacctggaaatgattttgcacacgcgtggcatccgcaacatcgtgctgaccggcatcaccaccgacgtctgcgtgcacaccaccatgcgcgaagccaacgaccgcggcttcgaatgcgtgatgctgtcggactgctgcggtgcgaccgaccacaataaccacctggcagcgctgtccatgatcaagatgcagggcggcgtgttcggtgccgtctccgattcggcggcactgatcgatgtgatcggggcctga

SEQ ID NO: 14 - protein, 231 aa, Herbaspirillum sp.
MPELFIKAEPYAWPYDGALTPANTALIVIDMQTDFCGIGGYVDKMGYDLSLTRAPIEPIKRVLAAMRAGGYTIIHTREGHRPDLSDLPANKRWRSRQIGTNGVGIGDAGPCGRILVRGEPGWEIIPELAPIAGEIIIDKPGKGSFCATDLEMILHTRGIRNIVLTGITTDVCVHTTMREANDRGFECVMLSDCCGATDHNNHLAALSMIKMQGGVFGAVSDSAALIDVIGA

SEQ ID NO: 15 - DNA, 30 bp, Artificial Sequence
  primer sequence CAH001
aaatgaacgaatcaaattaacaaccatagg

SEQ ID NO: 16 - DNA, 24 bp, Artificial Sequence
  primer sequence CAH002
caccggacattacaaatgcaatcc

SEQ ID NO: 17 - DNA, 22 bp, Artificial Sequence
  primer sequence CAH003
ccatacctccttctgtaccacc

SEQ ID NO: 18 - DNA, 68 bp, Artificial Sequence
  primer sequence CAH004
ataaccataaccaagtaatacatattcaaactagtgccaccatgcgtactacctccgttggtgtgttc

SEQ ID NO: 19 - DNA, 73 bp, Artificial Sequence
  primer sequence CAH005
attaaaaaactatatcaattaatttgaattaacgcggccgcttaatcagacaatctggcgatcacagcgactg

SEQ ID NO: 20 - DNA, 72 bp, Artificial Sequence
  primer sequence CAH006
ataaccataaccaagtaatacatattcaaactagtgccaccatgcctattgctaaagttcataggatttcag

SEQ ID NO: 21 - DNA, 70 bp, Artificial Sequence
  primer sequence CAH007
attaaaaaactatatcaattaatttgaattaacgcggccgcttaacctctatctacaattacagcgactg

SEQ ID NO: 22 - DNA, 19 bp, Artificial Sequence
  primer sequence CAH008
gaaaacgtggcatcctctc

SEQ ID NO: 23 - protein, 16 aa, Artificial Sequence
  Consensus Amino Acid Sequence
  MISC_FEATURE (1)..(1): Xaa is Tyr or Phe
  MISC_FEATURE (2)..(2): Xaa is any amino acid
  MISC_FEATURE (6)..(6): Xaa is any amino acid
  MISC_FEATURE (8)..(8): Xaa is His or Asn
  MISC_FEATURE (12)..(12): Xaa is any amino acid
  MISC_FEATURE (16)..(16): Xaa is Pro or Ser
XXSGGXEXQGPXGGGX